(19) United States
(12) Patent Application Publication
(10) Pub. No.: US 2024/0298987 A1
(43) Pub. Date: Sep. 12, 2024

(54) Compounds For Molecular Imaging of Collagen Turnover and Methods Using Same

(71) Applicants: Yale University, New Haven, CT (US); The United States Government as represented by the Department of Veterans Affairs, Washington, DC (US)

(72) Inventors: Mehran Sadeghi, Easton, CT (US); Mani Salarian, Hamden, CT (US); Kiran Gona, New Haven, CT (US); Jakub Toczek, New Haven, CT (US)

(21) Appl. No.: 18/573,604

(22) PCT Filed: Jun. 22, 2022
(86) PCT No.: PCT/US2022/073093

§ 371 Date: Dec. 22, 2023

Related U.S. Application Data
(60) Provisional application No. 63/213,850, filed on Jun. 23, 2021.

(51) Int. Cl. A61B 6/00; A61B 5/055; A61B 6/03; A61B 6/42; A61K 45/06; A61K 51/08; C07K 4/00

(52) U.S. Cl. CPC A61B 6/481; A61B 5/055; A61B 6/032; A61B 6/037; A61B 6/4241; A61K 45/06; A61K 51/088; C07K 4/00; A61K 2123/00

(57) ABSTRACT

In one aspect, the present disclosure relates to an imaging agent comprising a detectable moiety covalently bound or coordinated to a “Moiety A” selected from a peptide, a chelator, or an organic compound comprising a leaving atom or a leaving group that can be substituted with a radioisotope. “Moiety A” is covalently bound to a flexible linker which is further covalently bound to a polypeptide of between 2 and 20 glycine-proline-hydroxyproline repeats. In certain embodiments, the detectable moiety comprises a radioisotope or metal. In another aspect, the disclosure relates to a method of using the imaging agents of the present disclosure to detect collagen turnover in a subject.

### CROSS-REFERENCE TO RELATED APPLICATIONS

This application claims priority under 35 U.S.C. § 119(e) to U.S. Provisional Patent Application No. 63/213,850, filed Jun. 23, 2021, which is hereby incorporated by reference in its entirety herein.

### STATEMENT REGARDING FEDERALLY SPONSORED RESEARCH

This invention was made with government support under HL138567 and HL007950 awarded by National Institutes of Health and I01 BX004038 awarded by U.S. Department of Veterans Affairs. The government has certain rights in the invention.

### SEQUENCE LISTING

The ASCII text file named “047162-7322WO1 Sequence Listing,” created on Jun. 4, 2021, comprising 2.3 Kbytes, is hereby incorporated by reference in its entirety

### BACKGROUND OF THE DISCLOSURE

Fibrosis a major pathological process in many diseases, from cardiomyopathy to pulmonary fibrosis, liver fibrosis, cirrhosis, scleroderma, aortic aneurysm, and cancer. The process of fibrogenesis in tissues is triggered by tissue injury, which can lead to dysregulated collagen synthesis and deposition. The hallmark of collagen structure is a triple helix, comprising a right-handed helix of 3 α-chains. α-chains are formed by repetitive Gly-X-Y (where X and Y are frequently proline and hydroxyproline) tri-peptide motifs, which self-assemble to form (pro)collagen fibers. Collagen is initially synthesized as procollagen, which is a precursor molecule with C- and N-terminal propeptides flanking the Gly-X-Y motifs. Following translation, the procollagen α-chains are imported into the endoplasmic reticulum, where they assemble into triple helix procollagen. Procollagen undergoes proteolytic cleavage to form collagen following its export into extracellular space, where it assembles into fibrils and higher-ordered structures. Accordingly, in the mature collagen fibers, the α-chains are highly organized. Single stranded α-chains are present only during collagen synthesis in the cells, and when collagen is undergoing degradation, e.g., by matrix metalloproteinases and cathepsins, in the extracellular space.

Structural imaging modalities, such as magnetic resonance imaging (MRI) and computed tomography (CT), provide a snapshot of the tissue structure at a given point without any information on disease activity, which is the target of therapeutic interventions to prevent progression and promote regression of fibrosis. In addition, there is some lag before the effect of therapeutic interventions is reflected on the tissue structure, and hence becomes detectable by CT or MRI. Therefore, novel non-invasive quantitative tools are needed to characterize fibrosis, detect matrix turnover, select the patients for emerging therapies, track the effect of therapeutic interventions, and/or improve prognosis.

There is a need in the art for novel imaging agents that can detect collagen turnover associated with collagen synthesis or degradation. There is also a need in the art for a novel method of monitoring collagen turnover in a subject using the imaging agent. The present invention satisfies these unmet needs.

### BRIEF SUMMARY OF THE DISCLOSURE

In one aspect, the invention provides a compound of formula (I), or a salt, solvate, or derivative thereof, wherein the substituents in (I) are defined elsewhere herein:

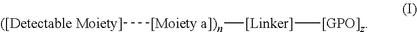

The invention further provides a method of imaging collagen turnorver in a subject, the method comprising administering to the subject a compound of formula (I), as defined elsewhere herein, and detecting a signal from the compound of formula (I) within the subject.

In certain embodiments, the signal is detected using single photon emission computed tomography (SPECT) imaging, positron emission tomography (PET), magnetic resonance imaging (MRI), magnetic resonance spectroscopic imaging (MRSI), fluorescence imaging, or a combination thereof.

In certain embodiments, the subject has fibrosis or is suspected of having fibrosis associated with a fibrotic disease or disorder. In certain embodiments, the fibrotic disease or disorder is pulmonary fibrosis, liver fibrosis, kidney fibrosis, aortic aneurysms, myocardial infarction, cardiomyopathy, scleroderma, heart failure, or a combination thereof.

In certain embodiments, the method further comprises administering a treatment for the fibrotic disease or disorder to the subject before administering the compound of formula (I).

In certain embodiments, the method further comprises administering a treatment for the fibrotic disease or disorder to the subject after administering the compound of formula (I). In certain embodiments, the efficacy of a treatment is evaluated by comparing a detected a signal from the compound of formula (I) administered before administration of a treatment and a detected signal from the compound of formula (I) administered after administration of a treatment.

### BRIEF DESCRIPTION OF THE DRAWINGS

The following detailed description of exemplary embodiments of the invention will be better understood when read in conjunction with the appended drawings. For the purpose of illustrating the invention, non-limiting embodiments are shown in the drawings. It should be understood, however, that the invention is not limited to the precise arrangements and instrumentalities of the embodiments shown in the drawings.

FIG. 1 depicts the ^{99m}Tc-labeled GPO tracers with ability to bind single stranded collagen. The targeting moiety consists of multiple GPO repeats linked to poly-His for site-specific labeling through a flexible linker.

FIGS. 2A-2B depict characterization of the His6-(GPO)₉ and control peptides. FIG. 2A: MALDI spectrum of synthesized His6-(GPO)₉ peptide. FIG. 2B: Circular dichroism spectroscopy of the His6-(GPO), peptide and the control His6-(G₉P₉O₉) peptide.

FIGS. 3A-3B depict TLC and HPLC characterization of ^{99m}Tc-His6-(GPO)₉ and related compounds. FIG. 3A: Radiochemical purity of ^{99m}Tc-His6-(GPO)₉ assessed with radio-TLC. FIG. 3B: Radiochemical purity of ^{99m}Tc-His6-(GPO) assessed with radio-HPLC.

FIGS. 4A-4B depict in vivo stability of ^{99m}Tc-His6-(GPO), following intravenous injection to mice. FIG. 4A: In vivo stability of the tracer at 0 minutes, 30 minutes, and 2 hr post injection (p.i.) in the bouse blood assessed with radio-HPLC. FIG. 4B: In vivo stability of the tracer at 0, 2, and 3 hr post injection (p.i.) in the mouse urine assed with radio-HPLC.

FIGS. 5A-5B depict ^{99m}Tc-His6-(GPO)₉ and control ^{99m}Tc-His6-(G₉P₉O₉) blood levels and biodistribution following intravenous injection to mice assessed by gamma well counting. ID: injected dose, pAT: peri-aortic adipose tissue, SG: salivary glands, WAT: white adipose tissue. FIG. 5A: Blood levels at 2 hours p.i. FIG. 5B: Biodistribution at 2 hours p.i.

FIGS. 6A-6D depict ex vivo studies of ^{99m}Tc-His6-(GPO), in a mouse model of abdominal aortic aneurysm (AAA). FIGS. 6A-6B: Ex vivo autoradiography of abdominal aortic aneurysm (AAA) induced by aminopropionitrile fumarate salt (BAPN)/Elastase using ^{99m}Tc-His6-(GPO)₉ compared to control tracer, ^{99m}Tc-His6-(G₉P₉O₉). FIG. 6C: Blood levels of ^{99m}Tc-His6-(GPO), and control tracer in mice with AAA, 2 h post injection. FIG. 6D: ^{99m}Tc-His6-(GPO), and control tracer uptake in aneurysm and descending thoracic aorta (DTA) based on autoradiography images.

FIGS. 7A-7D show molecular imaging studies of ^{99m}Tc-His6-(GPO), in a mouse model of AAA using SPECT/CT. FIGS. 7A-7B: SPECT/CT images acquired at 2 hours post injection of ^{99m}Tc-His6-(GPO), and ^{99m}Tc-His6-(G₉P₉O₉) in mice with AAA induced by BAPN/Elastase 30 days after surgery. FIG. 7C: Immunofluorescence microcopy images of aneurysm sections stained with single stranded collagen (left) compared to normal aorta. FIG. 7D: ^{99m}Tc-His6-(GPO), and control tracer uptake in aneurysm wall based on SPECT/CT images (left) compared to uptake in the tissues based on gamma counting (right).

FIG. 8 shows that collagen is initially synthesized as procollagen, a precursor molecule with C- and N-terminal propeptides flanking the Glycine-X-Y motifs, where X and Y are frequently proline and hydroxyproline. The procollagen α-chains are imported into the endoplasmic reticulum (1), where they assemble into triple helix procollagen (2). This process is initiated by the C-terminal propeptide. Procollagen is exported into the extracellular space (3), where it undergoes enzymatic cleavage of pro-peptides to form collagen (4). Cross-linking assembles collagen into fibrils and higher-ordered structures (5). Collagen degradation of mature collagen (6) by proteolytic enzymes, such as matrix metalloproteinases, generates single stranded α chains.

FIG. 9 shows examples of histologic images of an infarcted mouse heart at 1 week after LAD ligation demonstrating the presence of scar (arrow) by Masson's trichrome staining, collagen by Sirius red, and single-stranded collagen (fluorescence) by R-collagen hybridizing peptide (CHP) staining, where nuclei are stained with DAPI.

FIGS. 10A-10B depict in vivo studies of the ^{99m}Tc-His6-(GPO), and control imaging agents. FIG. 10A: Examples of CT and SPECT/CT images of a mouse with MI (top) or control tracer, ^{99m}Tc-His6-(G₉P₉O₉) (bottom) on subsequent days are shown at 2 hr p.i. Arrows point to areas with maximum tracer uptake in the infarct area. ID: injected dose. FIG. 10B: Quantification of tracer uptake in the left ventricle wall.

FIGS. 11A-11B depict in vivo studies of ^{99m}Tc-His6-(GPO)₉ in mice at 5 days after LAD occlusion or sham operation. FIG. 11A: Examples of SPECT/CT images acquired at 2 hours post injection of ^{99m}Tc-His6-(GPO), in mice at 5 days after LAD occlusion (right) and sham operation (left) demonstrating uptake of the tracer in the anterior and lateral myocardium (orange arrows). Cutaneous uptake, probably related to surgery, is observed in both animals (arrows). The SPECT signal in the sham-operated animal is enhanced to better visualize the skin uptake. FIG. 11B: Quantification of tracer uptake in the infarct and remote zones.

FIGS. 12A-12C depict studies from a mouse model of cardiac interstitial fibrosis. FIG. 12A: Mean gradient across the aortic arch constriction site in animals undergoing transaortic interstitial fibrosis (TAC) or sham operation. FIG. 12B: Examples of end diastole (ED) and end systole (ES) 2D echo images at 4 weeks after surgery in TAC and sham-operated animals. FIG. 12C: Left ventricle (LV) ejection fraction before and at 4 weeks after TAC.

FIGS. 13A-13B depict studies of ^{99m}Tc-His6-(GPO)₉ uptake in remodeled myocardium at 4 weeks after TAC. FIG. 13A: Examples of contrast enhanced CT (left), microSPECT (middle) and fused SPECT/CT images (right) on images acquired at 2 hours post injection of ^{99m}Tc-His6-(GPO)₉ (top row) and control ^{99m}Tc-His6-(G₉P₉O₉) (bottom row) at 4 weeks after TAC demonstrating patchy uptake of the targeted tracer in the myocardium (three arrows above bottom arrow). Uptake in the lungs is at least in part non-specific and may be related to pulmonary edema. Uptake in the sternum (bottom arrow) appears to be specific. FIG. 13B: Quantification of LV signal.

FIGS. 14A-14B depict studies of ^{99m}Tc-His6-(GPO)₉ uptake in a mouse model of lung fibrosis induced by bleomycin, FIG. 14A: Examples of lung H&E, Sirius Red (collagen) and R-CHP (single stranded collagen, with nuclei stained with DAPI) staining in control and at 4 weeks post-bleomycin exposure, demonstrating the development of fibrosis. FIG. 14B: ^{99m}Tc-His6-(GPO)₉ uptake in the right (R) lung and the heart evaluated by gamma counting at 2 hr post-intravenous tracer administration. ID: injected dose.

FIGS. 15A-15B depict blood levels and biodistribution following intravenous injection to mice of ^{99m}Tc-His3Glu3-(GPO), in comparison with ^{99m}Tc-His6-(G₉P₉O₉). FIG. 15A: Blood levels of both tracers assessed by gamma well counting. FIG. 15B: Biodistribution of both tracers at 2 hr p.i. assessed by gamma well counting

### DETAILED DESCRIPTION OF THE DISCLOSURE

Many existing gaps in the traditional approach to detecting fibrosis may be addressed by molecular imaging. Molecular imaging modalities that rely on radiotracers, i.e., single photon emission computed tomography (SPECT) and positron emission tomography (PET), are highly sensitive and can provide quantitative data to track the disease process in vivo. In recent years, several new tracers have been introduced to detect fibrosis. However, these agents target mature collagen, integrins, or fibroblasts. Therefore, a major shortcoming of these agents is that they do not distinguish between established disease and ongoing matrix remodeling which accompanies active fibrogenesis and resolution of fibrosis.

The present disclosure provides in one aspect imaging agents for the in vivo detection of collagen turnover in fibrosis. Without wishing to be limited by theory, it was hypothesized that the self-assembly of α-chains into a triple helix could be exploited to track matrix remodeling in fibrosis. Therefore, imaging agents were developed that adopt an α-helix confirmation and bind single stranded collagen fibers associated with matrix remodeling. In certain embodiments, the imaging agent comprises a detectable moiety which is covalently bound or coordinated to the rest of the imaging agent. In certain embodiments, the detectable moiety is covalently bound or coordinated to a “Moiety A” of the rest of the imaging agent. In some embodiments, the rest of the imaging agent comprises a “Moiety A” covalently linked via a flexible linker to a GPO polypeptide of repeating glycine, proline, and hydroxyproline residues. In certain embodiments, the detectable moiety is a radioisotope. In other embodiments, the detectable moiety is a metal. In some embodiments, the radioisotope is ^{99m}Tc. In certain embodiments, “Moiety A” is a peptide, a chelator, or an organic compound comprising a leaving atom or a leaving group that can be substituted with a radioisotope. In some embodiments, “Moiety A” is between 2 and 10 histidine residues. In certain embodiments, the flexible linker is GGG. In certain embodiments, the imaging agent is ^{99m}Tc-His6-(GPO)₉.

The present disclosure further provides methods of using the imaging agents to detect collagen turnover in a subject. In some embodiments, the subject has fibrosis or is suspected of having fibrosis. In certain embodiments, the subject has fibrosis or is suspected of having fibrosis associated with a fibrotic disease or disorder. In some embodiments, the method comprises administering an imaging agent disclosed herein to the subject and detecting a signal from the imaging agent. In certain embodiments, the step of detecting a signal from the imaging agent comprises detecting a signal from the imaging agent that has bound to single stranded collagen. In some embodiments, the signal from the imaging agent is detected by SPECT. In other embodiments, the signal from the imaging agent is detected by MRI, CT, or PET.

In certain embodiments, the method further comprises the step of administering a treatment for a fibrotic disease or disorder to the subject. The treatment can be administered to the subject either before or after the steps of the above method. In some embodiments, the treatment is administered to the subject before the steps of the above method and the step of detecting a signal from the imaging agent can be used to determine the effectiveness of the treatment. In other embodiments, the treatment is administered to the subject after the steps of the above method. In certain embodiments wherein the treatment is administered to the subject after the steps of the above method, the method further comprises a second administration of the imaging agent and detecting the signal from the imaging agent. In certain embodiments, the step of detecting the signal from the imaging agent can be used to determine the effectiveness of the treatment.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, selected methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “about” as used herein when referring to a measurable value such as an amount, a temporal duration, and the like, is meant to encompass variations of ±20% or ±10%, more preferably ±5%, even more preferably #1%, and still more preferably #0.1% from the specified value, as such variations are appropriate to perform the disclosed methods.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)—CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C═C(CH₃), and —CH₂C≡C(CH₂CH₃) among others.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In some instances, hyperproliferative disorders are referred to as a type of cancer including but not limited to primary or metastatic melanoma, thymoma, lymphoma, sarcoma, lung cancer, liver cancer, non-Hodgkin's lymphoma, Hodgkin's lymphoma, leukemias, uterine cancer, cervical cancer, bladder cancer, kidney cancer and adenocarcinomas such as breast cancer, prostate cancer, ovarian cancer, pancreatic cancer, and the like.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

The term “heteroaryl” as used herein refers to aromatic ring compounds containing 5 or more ring members, of which, one or more is a heteroatom such as, but not limited to, N, O, and S: for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridavinyl (3-pyridavinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl. 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “organic group” as used herein refers to any carbon-containing functional group. Examples can include an oxygen-containing group such as an alkoxy group, aryloxy group, aralkyloxy group, oxo(carbonyl) group: a carboxyl group including a carboxylic acid, carboxylate, and a carboxylate ester: a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include; sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soy bean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, PA), which is incorporated herein by reference.

The terms “patient,” “subject,” or “individual” are used interchangeably herein, and refer to any animal, or cells thereof whether in vitro or in situ, amenable to the methods described herein. In a non-limiting embodiment, the patient, subject, or individual is a human.

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound of the disclosure (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

### Compounds and Compositions

In one aspect, the present disclosure relates to an imaging agent comprising a detectable moiety covalently bound or coordinated to the rest of the imaging agent. In certain embodiments, the detectable moiety is covalently bound or coordinated to a “Moiety A” of the rest of the imaging agent. In some embodiments, the rest of the imaging agent comprises a “Moiety A” covalently bound to a flexible linker which is covalently bound to a glycine-proline-hydroxyproline (GPO) polypeptide. In certain embodiments, “Moiety A” is a peptide. In other embodiments, “Moiety A” is a chelator. In yet other embodiments, “Moiety A” is an organic compound comprising a leaving atom or a leaving group that can be substituted with a radioisotope. In certain embodiments, the imaging agent targets single stranded collagen. In certain embodiments, the imaging agent binds single stranded collagen. In certain embodiments, the imaging agent adopts an α-helix confirmation.

The detectable moiety can be any moiety that is used for single emission computed tomography (SPECT), positron emission tomography (PET), computed tomography (CT), magnetic resonance imaging (MRI), magnetic resonance spectroscopic imaging (MRSI), fluorescence imaging, and combinations thereof.

In certain embodiments, the detectable moiety is a fluorescent dye. In other embodiments, the detectable moiety is a nanoparticle. In yet other embodiments, the detectable moiety is a radioisotope. Exemplary radioisotopes include, but are not limited to, ³H, ¹¹C, ¹³C, ¹³N, ¹⁵O, ¹⁸F, ³²P, ³⁵S, ^{99m}Tc, ¹²³I, ⁶⁴Cu, ⁶⁷Ga, ⁶⁸Ga, and ¹¹¹In. In certain embodiments, the detectable moiety is ^{99m}Tc. In yet other embodiments, the detectable moiety is a metal. Exemplary metals include, but are not limited to, Gd³⁺, Fe³⁺, Mn²⁺, and Mn³⁺. In some embodiments, the radioisotope or metal comprises one or more ancillary ligands coordinated to the radioisotope or metal to satisfy its valency. Exemplary ancillary ligands include, but are not limited to, F⁻, Cl⁻, Br⁻, I⁻, CN⁻, ⁻OH, CO, NO₂⁻, and H₂O. In some embodiments, the imaging agent comprises more than one detectable moiety.

The “Moiety A” can be any moiety known to a person of skill in the art to bind to the detectable moiety. In embodiments wherein the detectable moiety comprises a radioisotope or a metal, the detectable moiety is noncovalently bound to “Moiety A”. In some embodiments, the detectable moiety is noncovalently bound to “Moiety A” via electrostatic interactions. In certain embodiments, the imaging agent comprises more than one “Moiety A.” In some embodiments, the imaging agent comprises both more than one detectable moiety and more than one “Moiety A.”

In some embodiments wherein the detectable moiety is a radioisotope or a metal, “Moiety A” is a protein or peptide capable of binding to the radioisotope or metal. In some embodiments, one or more amino acid side chains of the protein or peptide noncovalently bind to the radioisotope or metal. In certain embodiments, the peptide comprises one or more histidine residues or derivatives thereof. In certain embodiments, the peptide consists of between 2 and 20 repeating histidine residues. In certain embodiments, the peptide consists of 6 histidine residues. In certain embodiments, one or more of the histidine residues coordinates to the detectable moiety. In some embodiments, one or more of the histidine imidazole side chains coordinates to the detectable moiety. In certain embodiments, the detectable moiety is ^{99m}Tc and three histidine side chains coordinate to ^{99m}Tc. In some embodiments, ^{99m}Tc is further coordinated to three ancillary carbonyl ligands. In other embodiments, the peptide comprises histidine and glutamic acid. In certain embodiments, the peptide is (HisGlu)₃. In certain embodiments, one or more of the histidine residues of (HisGlu)₃ coordinates to the detectable moiety. In some embodiments, one or more of the histidine imidazole side chains of (HisGlu)₃ coordinates to the detectable moiety. In yet other embodiments, the peptide comprises glycine and cysteine. In certain embodiments, the peptide is (Gly)_{w}Cys, wherein w is an integer from 0 to 20.

In other embodiments wherein the detectable moiety is a radioisotope or a metal, “Moiety A” is an organic compound. In certain embodiments, the organic compound is a chelator. In certain embodiments, the chelator is a polyaminopolycarboxylato-based chelator. Exemplary chelators include, but are not limited to, diethylenetriaminepentaacetic acid (DTPA) 1,4,7,10-tetraazacyclododecane-1,4,7,10)-tetraacetic acid (DOTA), 1,4,7,10-tetraazacyclododececane, 1-(glutaric acid)-4,7,10-triacetic acid (DOTAGA), ethylenediaminetetraacetic acid (EDTA), 1,4,7-triazacyclononane-triacetic acid (NOTA), 1,4,7-triazacyclononane, 1-glutaric acid-4,7-acetic acid (NODAGA), 1,4,8,11-tetraazacyclotetradecane-1,4,8,11-tetraacetic acid (TETA), and combinations thereof. In other embodiments, the organic compound is a molecule with a leaving atom or a leaving group that can be substituted with a radioisotope. Exemplary molecules include, but are not limited to, aryltrifluoromethane sulfonates or alkyltrifluoromethane sulfonates.

In certain embodiments, “Moiety A” is covalently bound to the flexible linker. In certain embodiments wherein “Moiety A” is an organic compound, one or more of the atoms on the organic compound forms a covalent bond to the flexible linker. In certain embodiments wherein “Moiety A” is a protein or peptide, the C-terminus of the protein or peptide is covalently bound to the flexible linker. In other embodiments wherein “Moiety A” is a protein or peptide, the N-terminus of the protein or peptide is covalently bound to the flexible linker.

In certain embodiments, ^{99m}Tc is coordinated to “Moiety A” as ^{99m}TcO₄⁻ using a standard technetium tricarbonyl conversion technique. Therefore, in some embodiments, the ^{99m}Tc coordinated to “Moiety A” is further coordinated to three ancillary carbonyl ligands to satisfy its valency, thus forming ^{99m}Tc(CO)₃⁺. In certain embodiments, the detectable moiety coordinated to “Moiety A” can be detected by SPECT.

In other embodiments, the detectable moiety covalently bound to “Moiety A” can be detected by MRI. Therefore, some embodiments, the detectable moiety comprises a Gd³⁺, Fe³⁺, Mn²⁺, or Mn³⁺ noncovalently bound to a chelator “Moiety A” such that the detectable moiety coordinated or covalently bound to “Moiety A” can be detected using MRI.

The flexible linker can be any linker that acts to prevent interference between the detectable moiety and the GPO polypeptide-single stranded collagen interaction. In some embodiments, the flexible linker is covalently bound to “Moiety A.” In some embodiments, the flexible linker is covalently bound to the GPO polypeptide. In some embodiments wherein “Moiety A” is a protein or peptide, the flexible linker is covalently bound to the C-terminus of the protein or peptide. In other embodiments wherein “Moiety A” is a protein or peptide, the flexible linker is covalently bound to the N-terminus of the protein or peptide. In certain embodiments, the flexible linker is covalently bound to a side chain of an amino acid in the protein or peptide “Moiety A”. In certain embodiments, the flexible linker is covalently bound to a side chain of an amino acid in the GPO polypeptide.

In certain embodiments, the flexible linker is a peptide linker. In some embodiments, the N-terminal end of the peptide linker is covalently bound to the C-terminal end of the GPO polypeptide. In other embodiments, the C-terminal end of the peptide linker is covalently bound to the N-terminal end of the GPO polypeptide. In certain embodiments, an amino acid side chain can serve as the peptide linker. In certain embodiments, the peptide linker is a polypeptide comprising between 1 and 20 natural or unnatural amino acid residues. In certain embodiments, the peptide linker consists of between 1 and 20 glycine residues. In certain embodiments, the peptide linker consists of between 1 and 20 serine residues. In certain embodiments, the peptide linker has the formula [G]ₓ[S]_{y}, wherein x and y are each independently an integer selected from 1 to 20. In some embodiments, the peptide linker is selected from the group consisting of GGG, GGSGG (SEQ ID NO:1), GSGS (SEQ ID NO:2), and combinations thereof.

In other embodiments, the flexible linker is a small cyclic or acyclic organic molecule that can include at least one functional group selected from the group consisting of ethers, ketones, amides, alkyne, azide, amine, isothiocyanate, and combinations thereof. In some embodiments, one or more of the functional groups on the cyclic or acyclic organic molecule reacts with “Moiety A” and/or reacts with the GPO polypeptide to form a covalent bond between the small cyclic or acyclic organic molecule, “Moiety A”, and/or the GPO polypeptide.

In yet other embodiments, the flexible linker is selected from polyethylene glycol and polypropylene glycol. In some embodiments, the polyethylene glycol or polypropylene glycol flexible linker forms a covalent bond with “Moiety A” and/or the GPO polypeptide. In certain embodiments, the polyethylene glycol or polypropylene glycol flexible linker has a molecular weight of between about 0.25 to 30 kDa. In certain embodiments, the polyethylene glycol or polypropylene glycol flexible linker is a straight chain polyethylene glycol or polypropylene glycol, a branched polyethylene glycol or polypropylene glycol, or a combination thereof. In some embodiments, the polyethylene glycol or polypropylene glycol flexible linker comprises one or more functional groups that form a covalent bond with “Moiety A” and/or the GPO polypeptide. In certain embodiments, the one or more functional groups are selected from selected from the group consisting of ethers, ketones, amides, alkyne, azide, amine, isothiocyanate, and combinations thereof. In yet other embodiments, the flexible linker is a hydrocarbon linker. In some embodiments, the flexible linker is a C₁-C₂₀ hydrocarbon linker. In some embodiments, the hydrocarbon flexible linker comprises one or more functional groups that form a covalent bond with “Moiety A” and/or the GPO polypeptide. In certain embodiments, the one or more functional groups are selected from selected from the group consisting of ethers, ketones, amides, alkyne, azide, amine, isothiocyanate, and combinations thereof.

The GPO polypeptide can comprise any number of glycine, proline, hydroxyproline residues, or derivatives thereof. In some embodiments, the number of glycine, proline, hydroxyproline residues, or derivatives thereof is selected such that the GPO polypeptide interacts with single stranded collagen but does not self-assemble or adversely affect tissue access and blood clearance. In certain embodiments, the GPO polypeptide comprises between 2 and 20 GPO repeats. In certain embodiments, the GPO polypeptide comprises 5, 7, 9, 11, or 13 GPO repeats. In certain embodiments, the GPO polypeptide is (GPO)₉ (SEQ ID NO:3). In certain embodiments, the C-terminal end of the GPO polypeptide is bonded to the flexible linker. In certain embodiments, one or more of the proline residues in the GPO polypeptide is a (2S,4S)-4-fluoroproline residue.

In some embodiments, the imaging agent is an compound of formula (I):

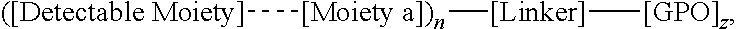

or a salt, solvate, or derivative thereof, wherein
each [Detectable Moiety] is independently a radioisotope or metal selected from the group consisting of ³H, ¹¹C, ¹³C, ¹³N, ¹⁵O, ¹⁸F, ³²P, ³⁵S, ^{99m}Tc, ¹²³I, ⁶⁴Cu, ⁶⁷Ga, ⁶⁸Ga, ¹¹¹In, Gd³⁺, Fe³⁺, Mn²⁺, and Mn³⁺, wherein [Detectable Moiety] is optionally coordinated to one or more ancillary ligands to satisfy its valency;
each [Moiety A] is independently selected from the group consisting of:
   a peptide comprising between 1 and 20 natural or unnatural amino acid residues,
   apolyaminopolycarboxylato-based chelator, and
   an organic compound comprising a leaving atom or a leaving group that can be substituted with a radioisotope:
[Linker] is selected from the group consisting of: a peptide comprising between 1 and 20 natural or unnatural amino acid residues, a small cyclic organic molecule, a small acyclic organic molecule, polyethylene glycol, polypropylene glycol, and a hydrocarbon chain;
[GPO] is glycine-proline-hydroxyproline, wherein each instance of proline is independently optionally (2S,4S)-4-fluoroproline;
n is an integer between 1 and 10;
z is an integer between 2 and 20; and
wherein [symbol] represents one or more coordinate covalent bonds, one or more covalent bonds, or a combination thereof;
and [symbol] represents one or more covalent bonds.

In certain embodiments, the one or more ancillary ligands coordinated to the [Detectable Moiety] are selected from the group consisting of F⁻, Cl⁻, Br⁻, I⁻, CN⁻, ⁻OH, CO, NO₂⁻, and H₂O.

In certain embodiments, n is 1. In other embodiments, n is greater than 1 and the imaging agent of formula (I) comprises more than one detectable moiety and more than one [Moiety A].

In certain embodiments, [Detectable Moiety] is ^{99m}Tc. In certain embodiments, [Detectable Moiety] is ^{99m}Tc coordinated to three ancillary CO ligands. In certain embodiments, ^{99m}Tc is coordinated to three ancillary CO ligands and forms three coordinate covalent bonds to [Moiety A].

[Moiety A] can be any “Moiety A” described elsewhere herein. In certain embodiments, [Moiety A] is a peptide comprising one or more histidine residues or derivatives thereof. In certain embodiments, [Moiety A] consists of between 2 and 20 repeating histidine residues. In certain embodiments, [Moiety A] consists of 6 histidine residues. In certain embodiments, one or more of the histidine residues of [Moiety A] coordinates to [Detectable Moiety]. In some embodiments, one or more of the histidine imidazole side chains of [Moiety A] coordinates to [Detectable Moiety]. In certain embodiments, [Detectable Moiety] is ^{99m}Tc and three histidine side chains of [Moiety A] coordinate to ^{99m}Tc.

[Linker] can be any flexible linker described elsewhere herein. In certain embodiments. [Linker] is a peptide comprising one or more glycine residues or derivatives thereof. In certain embodiments, [Linker] consists of between 1 and 10 repeating glycine residues. In certain embodiments, [Linker] consists of 3 glycine residues.

In certain embodiments, z is 5, 7, 9, 11, or 13. In certain embodiments, z is 9) and [GPO]9 contains 9 repeating G-P—O residues covalently bonded to each other. In some embodiments, one or more of the proline residues of [GPO] is (2S,4S)-4-fluoroproline. In certain embodiments, one or more of the proline residues of [GPO] is substituted with a fluorine atom.

In certain embodiments, the imaging agent of formula (I) comprises a ^{99m}Tc [Detectable Moiety], a histidine [Moiety A], a glycine [Linker], and a [GPO] polypeptide. In certain embodiments, the imaging agent has a ^{99m}Tc detectable moiety, a “Moiety A” of 6 histidine residues, a flexible linker of 3 glycine residues, and a GPO polypeptide of 9 repeating G-P—O residues, which is referred to herein as ^{99m}Tc-His6-(GPO)₉. In certain embodiments. ^{99m}Tc-His6-(GPO)₉ includes any ancillary ligands described elsewhere herein coordinated to ^{99m}Tc to satisfy its valency. In some embodiments, ^{99m}Tc-His6-(GPO)₉ includes three ancillary CO ligands coordinated to ^{99m}Tc to satisfy its valency.

In another aspect, the present invention relates to a composition comprising an imaging agent described elsewhere herein. In certain embodiments, the composition comprises a solvent. The solvent can be any organic or aqueous solvent known to a person of skill in the art. Exemplary organic solvents include, but are not limited to, methanol, ethanol, isopropanol, n-butanol, t-butanol, pentanes, hexanes, benzene, toluene, dichloromethane, chloroform, diethyl ether, dimethyl ether, ethyl acetate, dimethylformamide, and combinations thereof. Exemplary aqueous solvents include, but are not limited to, distilled water, deionized water, saline, Ringer's lactate solution, and combinations thereof. In certain embodiments, the solvent is selected from water for injection, saline solution, fixed oils, polyethylene glycols, glycerin, propylene glycol, and combinations thereof.

In certain embodiments, the composition comprises an anti-bacterial agent such as benzyl alcohol. In certain embodiments, the composition comprises an antioxidant such as ascorbic acid or sodium bisulfite. In certain embodiments, the composition comprises a buffer such as acetates, citrates, or phosphates. In certain embodiments, the composition comprises an agent for the adjustment of tonicity such as sodium chloride or dextrose. In certain embodiments, the pH of the composition can be adjusted with acids or bases, such as hydrochloric acid or sodium hydroxide.

In certain embodiments, the composition comprises an inactive ingredient. The inactive ingredient may be any inactive ingredient known to a person of skill in the art. In certain embodiments, the inactive ingredient is selected from the group consisting of excipients, diluents, fillers, binders, disintegrants, lubricants, colorants, preservatives, surfactants, stabilizers, viscosity increasing agents, sweeteners, and any combinations thereof. In certain embodiments, the inactive ingredient is a pharmaceutically acceptable carrier. Exemplary pharmaceutical carriers are described elsewhere herein.

### Methods

In another aspect, the present invention relates to a method of imaging collagen turnover in a subject, the method comprising: (a) administering an imaging agent disclosed herein to the subject; and (b) detecting a signal from the imaging agent.

In certain embodiments, the imaging agent is a compound of formula (I). The compound of formula (I) can be any compound of formula (I) disclosed elsewhere herein, or a salt, solvate, or derivative thereof. In certain embodiments, the compound of formula (I) has a [Detectable Moiety] of ^{99m}Tc. In some embodiments, [Detectable Moiety] is ^{99m}Tc which is coordinated to three ancillary CO ligands. In certain embodiments, the compound of formula (I) has a [Moiety A] of one or more histidine residues. In some embodiments, the compound of formula (I) has a [Moiety A] of six histidine residues. In some embodiments, the ^{99m}Tc [Detectable Moiety] is covalently coordinated to three histidine residues of [Moiety A]. In certain embodiments, the compound of formula (I) has [Linker] of one or more glycine residues. In certain embodiments, the compound of formula (I) has a [Linker] of three glycine residues. In certain embodiments, the compound of formula (I) comprises a [GPO] polypeptide having nine GPO repeats. In some embodiments, the compound of formula (I) is ^{99m}Tc-His6-(GPO)₉. In certain embodiments, the compound of formula (I) adopts an α-helix configuration.

The subject can be any subject in need of imaging of collagen turnover. In certain embodiments, the subject has fibrosis or is suspected of having fibrosis. In certain embodiments, the subject has fibrosis or is suspected of having fibrosis associated with a fibrotic disease or disorder. Exemplary fibrotic diseases or disorders include, but are not limited to, myocardial injury (including myocardial infarction, cardiomyopathy, calcific aortic valve disease and/or heart failure), pulmonary fibrosis, aortic aneurysms, interstitial lung disease, cancer, liver disease, kidney disease, scleroderma, rheumatoid arthritis, Crohn's disease, ulcerative colitis, myelofibrosis, lupus, or a combination thereof.

The imaging agent can be administered to the subject using any technique known to a person of skill in the art. Exemplary administration methods include, but are not limited to, oral administration and intravenous administration. In certain embodiments, the imaging agent is administered via an intravenous injection. In some embodiments, the imaging agent is injected at a site on the subject wherein collagen turnover is suspected. In some embodiments, the site wherein collagen turnover is suspected is a site of fibrosis in the subject. In some embodiments, the collagen turnover is associated with the presence of single stranded collagen. In certain embodiments, the imaging agent targets single stranded collagen and therefore can be injected at any site on the subject. Although not wishing to be limited by theory, it is believed that single stranded collagen α-chains are only present during collagen synthesis in cells and when collagen is undergoing degradation. In certain embodiments, the imaging agent is administered to the subject as a component of a composition. Other optional components of the composition are described elsewhere herein.

The signal from the imaging agent can be detected using any method known to a person of skill in the art for in vivo detection of the detectable moiety of the imaging agent. In certain embodiments, the imaging agent is detected by SPECT imaging. In other embodiments, the imaging agent is detected by MRI imaging. In yet other embodiments, the imaging agent is detected by CT imaging. In yet other embodiments, the imaging agent is detected by SPECT/CT or PET/CT imaging. In certain embodiments, a signal is detected when the imaging agent binds to single stranded collagen in the subject. In certain embodiments, the single stranded collagen is associated with collagen turnover in the subject. In certain embodiments, the collagen turnover is associated with fibrogenesis in the subject. Therefore, in some embodiments, the signal from the imaging agent can be used to diagnose fibrosis in the subject or to monitor the progression of fibrosis in the subject. In other embodiments, the collagen turnover is associated with the resolution of fibrosis in the subject.

Although not wishing to be limited by theory, it is believed that the imaging agents disclosed herein may be tailored such that they can detect different stages of fibrosis in the subject. Therefore, it is believed that imaging agents that are designed to be cell-penetrating will detect both the synthesis of mature collagen from three single stranded α-chains to a triple helix structure and the degradation of mature collagen fibers to single stranded α-chains. Conversely, it is believed that imaging agents that remain extracellular will target the resolution of fibrosis. In addition, there is collagen remodeling during both synthesis and degradation of mature collagen, wherein the remodeling process can be detected by the imaging agents disclosed herein.

In some embodiments, the method further comprises the step of administering a treatment for a fibrotic disease or disorder to the subject. In certain embodiments, the step of administering a treatment for a fibrotic disease or disorder to the subject precedes step (a) of administering an imaging agent disclosed herein to the subject. In other embodiments, the step of administering a treatment for a fibrotic disease or disorder to the subject follows step (b) of detecting a signal from the imaging agent. In some embodiments wherein the step of administering a treatment for a fibrotic disease or disorder to the subject follows step (b), the method further comprises the steps of: (c) administering imaging agent disclosed herein to the subject; and (d) detecting a signal from the imaging agent. Therefore, in some embodiments, the first administration (step (a)) and detection of the imaging agent (step (b)) can be used as a “baseline” of collagen turnover at a site of interest in the subject before administration of a treatment and the second administration (step (c)) and detection of the imaging agent (step (d)) can be used to monitor the effectiveness of the treatment. In certain embodiments, the intensity of the signal detected in step (b) can be compared to the intensity of the signal in step (d) to monitor the effectiveness of the treatment. In certain embodiments, when the imaging agent used in step (b) and in step (d) remains extracellular, an increased signal intensity in step (d) when compared to step (b) is indicative of a resolution of fibrosis in the subject and thus further indicates that the treatment is effective. In other embodiments, when the imaging agent used in step (b) and in step (d) remains extracellular, an decreased signal intensity in step (d) when compared to step (b) indicates that the treatment is not effective in resolving fibrosis in the subject.

The treatment can be any treatment or combination of treatments known or believed to be useful in treating a fibrotic disease or disorder. Exemplary fibrotic diseases or disorders are described elsewhere herein. Exemplary treatments for a fibrotic disease or disorder include, but are not limited to, modulators of the renin-angiotensin-aldosterone system (RAAS) such as ACE inhibitors, modulators of the TGF-β signaling pathway, proteases, implantable biomaterials, cell transplantation therapy, cell reprogramming, non-coding RNAs, epigenetic modifiers, and combinations thereof. In certain embodiments, the subject has or is suspected of having pulmonary fibrosis and is administered a treatment for pulmonary fibrosis. In certain embodiments, the treatment is a lifestyle change such as exercise or a healthy diet. In other embodiments, the treatment is supplemental oxygen. In yet other embodiments, the treatment is a drug therapy. Exemplary drug therapies for pulmonary fibrosis include, but are not limited to, nintedanib, pirfenidone, corticosteroids, mycophenolate mofetil, mycophenolic acid, azathioprine, and combinations thereof. In other embodiments, the subject has or is suspected of having fibrosis associated with myocardial injury and is administered a treatment for the myocardial injury. Exemplary myocardial injuries include, but are not limited to, heart failure, myocardial infarction, and cardiomyopathy. In certain embodiments, the treatment is a lifestyle change such as exercise or a healthy diet. In other embodiments, the treatment is a drug therapy. Exemplary drug therapies for myocardial injury include, but are not limited to, blood thinners, nitrates, beta blockers, calcium channel blockers, cholesterol-lowering therapies, ACE inhibitors, and combinations thereof. In certain embodiments, the subject has or is suspected of having liver fibrosis and is administered a treatment for liver fibrosis. In certain embodiments, the treatment is a lifestyle change such as exercise, a healthy diet, or avoiding alcohol. In other embodiments, the treatment is a drug therapy.

### Pharmaceutical Compositions and Formulations

The invention provides pharmaceutical compositions comprising at least one compound of the invention or a salt or solvate thereof, which are useful to practice methods of the invention. Such a pharmaceutical composition may consist of at least one compound of the invention or a salt or solvate thereof, in a form suitable for administration to a subject, or the pharmaceutical composition may comprise at least one compound of the invention or a salt or solvate thereof, and one or more pharmaceutically acceptable carriers, one or more additional ingredients, or any combinations of these. At least one compound of the invention may be present in the pharmaceutical composition in the form of a physiologically acceptable salt, such as in combination with a physiologically acceptable cation or anion, as is well known in the art.

In certain embodiments, the pharmaceutical compositions useful for practicing the method of the invention may be administered to deliver a dose of between 1 ng/kg/day and 100 mg/kg/day. In other embodiments, the pharmaceutical compositions useful for practicing the invention may be administered to deliver a dose of between 1 ng/kg/day and 1,000 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for nasal, inhalational, oral, rectal, vaginal, pleural, peritoneal, parenteral, topical, transdermal, pulmonary, intranasal, buccal, ophthalmic, epidural, intrathecal, intravenous, or another route of administration. A composition useful within the methods of the invention may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In certain embodiments, the compositions of the invention are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In certain embodiments, the compositions of the invention are formulated using one or more pharmaceutically acceptable excipients or carriers. In certain embodiments, the pharmaceutical compositions of the invention comprise a therapeutically effective amount of at least one compound of the invention and a pharmaceutically acceptable carrier. Pharmaceutically acceptable carriers, which are useful, include, but are not limited to, glycerol, water, saline, ethanol, recombinant human albumin (e.g., Recombumin®), solubilized gelatins (e.g., Gelofusine®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring, and/or fragrance-conferring substances and the like. They may also be combined where desired with other active agents. e.g., other analgesic, anxiolytics or hypnotic agents. As used herein, “additional ingredients” include, but are not limited to, one or more ingredients that may be used as a pharmaceutical carrier.

The composition of the invention may comprise a preservative from about 0.005% to 2.0% by total weight of the composition. The preservative is used to prevent spoilage in the case of exposure to contaminants in the environment. Examples of preservatives useful in accordance with the invention include but are not limited to those selected from the group consisting of benzyl alcohol, sorbic acid, parabens, imidurea and any combinations thereof. One such preservative is a combination of about 0.5% to 2.0% benzyl alcohol and 0.05-0.5% sorbic acid.

The composition may include an antioxidant and a chelating agent that inhibit the degradation of the compound. Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.01% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non-ionic surfactants. Known preservatives include, but are not limited to, methyl, ethyl, or n-propyl para-hydroxy benzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, ionic and non-ionic surfactants, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soy bean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration

Routes of administration of any of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic, generally recognized as safe (GRAS) pharmaceutically excipients which are suitable for the manufacture of tablets. Such excipients include, for example an inert diluent such as lactose: granulating and disintegrating agents such as cornstarch: binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation. Hard capsules comprising the active ingredient may be made using a physiologically degradable composition, such as gelatin. The capsules comprise the active ingredient, and may further comprise additional ingredients including, for example, an inert solid diluent such as calcium carbonate, calcium phosphate, or kaolin.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin from animal-derived collagen or from a hypromellose, a modified form of cellulose, and manufactured using optional mixtures of gelatin, water and plasticizers such as sorbitol or glycerol. Such soft capsules comprise the active ingredient, which may be mixed with water or an oil medium such as peanut oil, liquid paraffin, or olive oil.

For oral administration, the compounds of the invention may be in the form of tablets or capsules prepared by conventional means with pharmaceutically acceptable excipients such as binding agents: fillers: lubricants: disintegrates: or wetting agents. If desired, the tablets may be coated using suitable methods and coating materials such as OPADRY® film coating systems available from Colorcon, West Point, Pa. (e.g., OPADRY® OY Type, OYC Type, Organic Enteric OY-P Type, Aqueous Enteric OY-A Type, OY-PM Type and OPADRY® White, 32K18400). It is understood that similar type of film coating or polymeric products from other companies may be used.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface-active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds useful within the methods of the invention, and a further layer providing for the immediate release of one or more compounds useful within the methods of the invention. Using a wax/pH-sensitive polymer mix, a gastric insoluble composition may be obtained in which the active ingredient is entrapped, ensuring its delayed release.

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia): non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Additional Administration Forms

Additional dosage forms of this invention include dosage forms as described in U.S. Pat. Nos. 6,340,475, 6,488,962, 6,451,808, 5,972,389, 5,582,837, and 5,007,790.

Additional dosage forms of this invention also include dosage forms as described in U.S. Patents Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### EXPERIMENTAL EXAMPLES

The invention is further described in detail by reference to the following experimental examples. These examples are provided for purposes of illustration only, and are not intended to be limiting unless so specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Materials and Methods

### Peptide Synthesis and Radiolabeling

Peptides were synthesized using a peptide synthesizer, then they were radiolabeled with ^{99m}TcO₄⁻ in two steps based on previously reported procedures. In the first step, ^{99m}TcO₄⁻ was converted to ^{99m}Tc(CO)₃⁺ using an Isolink kit consisted of lyophilized formulation in an N₂-flushed 10-mL glass vial, containing 8.5 mg sodium tartrate (Na₂C₄H₄O₆), 2.85 mg sodium tetraborate (Na₂B₄O₇), 7.15 mg sodium carbonate (Na₂CO₃), and 4.5 mg sodium boranocarbonate (Na₂H₃BCO₂), then the peptide (50 μg) was site-specifically radiolabeled with ^{99m}Tc(CO)₃⁺ with heating at 60° C. for 40 min. Illustra NAP-5 columns (GE) was used to separate free ^{99m}Tc(CO)₃⁺ and ^{99m}TcO₄⁻ from the labeled peptide. Radio-high performance liquid chromatography (HPLC) and thin layer chromatography (TLC) was used to confirm radiochemical purity.

The above Methods section also applies to Examples 2 and 3.

### Circular Dichroism (CD)

Circular Dichroism (CD) spectra was collected on Chirascan™ spectrometer (Applied Photophysics) equipped with quartz cells (1 mm pathlength). Peptide solutions (400 μL, 150 μM in 1×PBS) were stored at 4° C. for at least 24 hr before measurement.

The above Methods section also applies to Examples 2 and 3.

### SPECT CT Imaging

Imaging was performed on a U-SPECT4CT system (MILabs) with hybrid multi-pinhole high resolution (0.4 mm) collimator as described. SPECT reconstruction was performed on an isotropic 0.125 mm voxel grid and reconstructed images was corrected for attenuation and scatter. Reconstructed images were analyzed with Slicer 3D (www.slicer.org).

### Animal Model of AAA

To generate BAPAN/Elastase-induced AAA, animals were treated with β-aminopropionitrile (BAPN, Sigma) 0.2% in drinking water. Two days after the start of BAPN administration, the animals underwent surgery. The infra-renal abdominal aorta was exposed, and porcine pancreatic elastase (6.67 mg/mL, 10 U/mg, MP Biomedical) was topically applied for 5 min. At 4-5 weeks after surgery, the animals underwent SPECT/CT imaging, followed by animal euthanasia and tissue harvesting.

### Tracer Biodistribution and Blood Clearance Thereof

The biodistribution and blood clearance of ^{99m}Tc-His6-(GPO), and ^{99m}Tc-His6-(G₉P₉O₉) were investigated in C57BL/6J mice following intravenous administration of each radiotracer (18.5±3.7 MBq). Serial blood samples were collected over a 2 h period, following which the animals were euthanized, different tissue samples and body fluids were collected and weighed, and their radioactivity was measured by gamma-well counting.

The above Methods section also applies to Examples 2 and 3.

### Ex Vivo Autoradiography

At 2 h post injection, the aorta was dissected from the surrounding tissues and placed on a phosphor screen (MultiSensitive Phosphor Screen, PerkinElmer) along with standard references of known activity for quantitative autoradiography. The phosphor screen was scanned with a phosphorimager (Typhoon Trio, GE Healthcare Life Sciences) to obtain digitalized images of radiotracer uptake, where focal tracer uptake was readily detectable in animals with AAA. Regions of interest (ROIs) were drawn over different segments of the aorta to quantify the ^{99m}Tc-His6-(GPO), and ^{99m}Tc-His6-(G₉P₉O₉) signals (Fiji/ImageJ. software, NIH).

### Tissue Analysis

Formalin-fixed samples were embedded in paraffin, cut into 5 μm-thick sections and stained with Sirius Red and Masson's Trichrome according to standard procedures. Sirius red-stained sections were analyzed using ImageJ to quantify the percentage of the slide stained in red. Collagen Hybridizing Peptide, Cy3 Conjugate (R-CHP, 3Helix) was used to stain for single stranded collagens. Briefly, a solution of the peptide with a concentration of 20 μM was heated to 80° C. for 5 min in a water bath in a sealed microtube followed by quenching in an ice-water bath for 15-90 s and subsequently applied to the tissues and incubated overnight at 4° C.

### Animal Model of Pulmonary Fibrosis

Bleomycin-induced pulmonary fibrosis was used as a model for imaging single stranded collagen. Bleomycin (2 U/kg) was instilled transorally into 6-8 week old C57Bl/6 mouse lungs under anesthesia. Animals were euthanized at the time point of interest and the lungs were perfused with PBS and collected for histology analysis.

### Animal Model of Myocardial Infarction

A mouse model of MI induced by LAD permanent ligation was used to assess the tracer in vivo properties. In this model, 8-10-week old male and female C57Bl/6J mice (Jackson Laboratory) underwent thoracotomy and permanent LAD coronary artery ligation. The procedure was performed under inhaled anesthesia with 0.5-2% isoflurane. Pre-emptive buprenorphine (0.05-0.1 mg/kg, IP) was given 20 minutes prior to surgery. Electrocardiogram (ECG) was monitored throughout the procedure. A midline cervical skin incision was performed, and a 20-G plastic catheter was inserted into the trachea and attached to a mouse ventilator. Ventilation was performed with a tidal volume of 200 μL and respiratory rate of 120 per minute. A skin incision was then made in the left mid-clavicular line between the 3rd and 4th ribs. LAD was ligated permanently with 8-0 non-absorbable suture under Leica M125 microscope to precisely occlude the LAD 2 mm from the left auricle. The ischemic heart area will have a pale color and the ECG will show significant ST-elevation after successful LAD ligation. Sham operation was performed with placement of an untied suture without coronary occlusion.

microSPECT CT Imaging

The animals were imaged on a dedicated small animal microSPECT/CT scanner (U-SPECT4CT system, MILabs) using a dedicated high sensitivity mouse multi-pinhole collimator (XMUS-M 2.0 mm ph). A 45 min-long list-mode acquisition centered on the Tc photopeak (140 keV±20%) started at 2 h after the injection of ˜1 mCi of ^{99m}Tc-His6-(GPO)₉. Images were reconstructed using the Similarity-Regulated Ordered Subsets Expectation Maximization (SROSEM) algorithm and a 0.4 mm isotropic voxel size.

### Example 1: Molecular Imaging of Collagen Degradation in Abdominal Aortic Aneurysm

Abdominal aortic aneurysm (AAA) is defined as an irreversible dilatation of the aorta with a diameter greater than 30 mm, which represents an increase of more than 50% compared to the normal aortic diameter. With disease progression and enlargement of aortic diameter, the risk of AAA rupture increases. Aortic rupture represents a life-threatening complication of aneurysms with an overall mortality rate of >90% in western countries. Several studies have identified distinct risk factors for aneurysm development. These include old age, male sex, cigarette smoking, obesity, high levels of low-density lipoprotein (LDL), and hypertension, which are all associated with a higher prevalence of AAAs. The prolonged course of subclinical asymptomatic disease opens a relatively long diagnostic window before life-threatening complications occur.

Molecular imaging has high potential to improve the characterization of AAAs, as well as to enable the development of novel prognostic parameters for the assessment of AAAs and to guide novel therapeutic approaches. In clinical practice, Ultrasound (US), magnetic resonance imaging (MRI), and computed tomography (CT) are currently performed for the identification and assessment of AAAs by measuring aortic diameters. However, the value of aortic diameter alone, as a diagnostic and prognostic parameter, is limited. Therefore, relying on AAA size alone is not sufficient for a reliable prediction of aneurysm progression and risk of rupture. New diagnostic tools are needed to improve the risk stratification of aortic aneurysms.

Molecular imaging techniques have the potential to improve not only imaging-based diagnosis and risk stratification but also the assessment of responses to therapy, enabling the detection of changes at a molecular/biological level before the onset of morphological changes. Such an approach surpasses what is currently achievable with traditional imaging modalities such as MRI and CT.

The hallmark pathology of AAA is a persistent proteolytic imbalance that results in excess extracellular matrix (ECM) destruction such as collagen and progressive weakening of the arterial wall. Furthermore, growth and rupture of AAAs result from increased collagen turnover. Herein, ^{99m}Tc-His6-(GPO)₉ was used to detect single stranded collagen by SPECT/CT in a murine AAA model.

### Example 2: Design of Novel Radiotracers

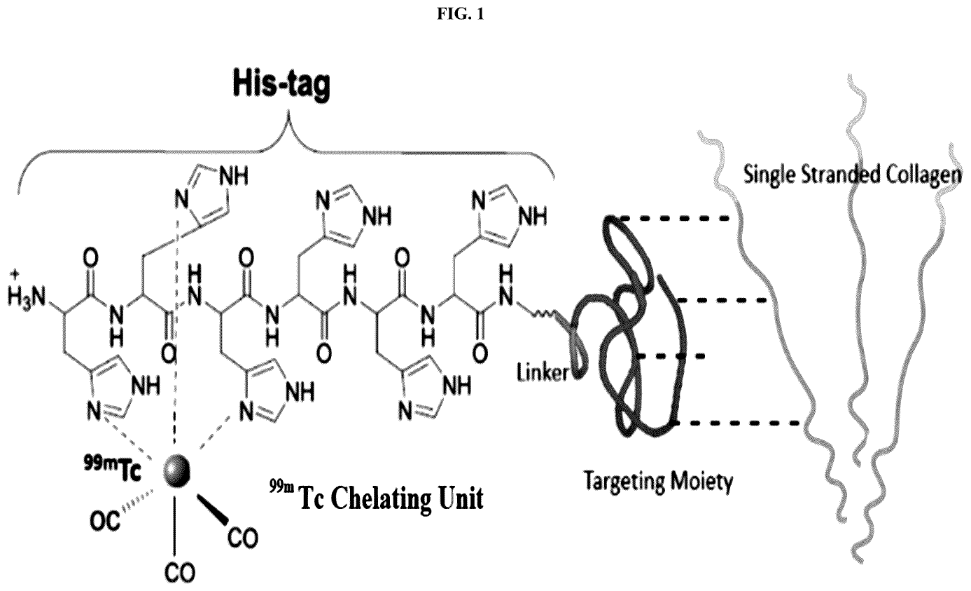

To access novel radiotracers that target single-stranded collagen, an imaging agent was designed comprising a N-terminal poly-histidine (His6) connected to a C-terminal targeting moiety comprising 9 Glycin-Proline-Hydroxyproline (GPO) repeats through a flexible 3 Glycine linker (^{99m}Tc-His6-(GPO)₉) (FIG. 1).

This rational modular design allows for rapid and efficient site-specific ^{99m}Tc-labeling using established methodology that has been shown to be safe in humans. Further, the use of a linker affords flexibility and distance to prevent interference with the GPO polypeptide-single stranded collagen interaction, while reducing spontaneous self-trimerization of the tracer. The radiotracer can also be modified to promote renal or hepatic clearance, as increases necessary.

In addition to facilitating site-specific labeling, His6 increases the GPO peptide stability by increasing its length to >30 amino acids and capping the free N-terminus, which may render the GPO peptide resistant to proteases specialized for proline containing peptides. The composition comprising this histidine-containing tag can be modified to modulate hepatic uptake of the tracer, if necessary.

Additionally, a compound with a similar structure which contains a scrambled GPO moiety (i.e., ^{99m}Tc-His6-G₉P₉O₉) was designed to serve as a control for the tracers described herein.

### Example 3: Peptide Synthesis and Helix Formation Capacity

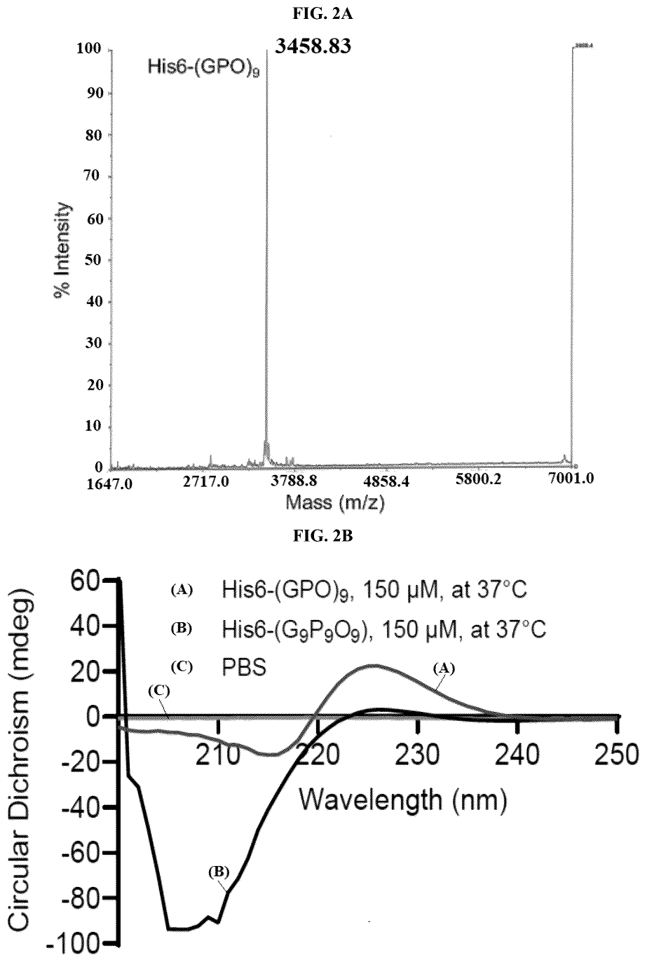

The peptide consisting of 6 His, 3 Gly and 9 GPO repeats (His6-(GPO)₉) was synthesized using Fmoc solid-phase peptide synthesis (SPPS) and its purity was confirmed with Matrix-Assisted Laser Desorption-Time of Flight (MALDI-TOF) mass spectrometry (FIG. 2A). The peptides helix-forming capacity were evaluated with circular dichroism (CD). As shown in FIG. 2B, His6-(GPO)₉ shows a positive peak near 225 nm and a negative peak near 215 nm which suggest a polyproline II helix conformation. For His6-(G₉P₉O₉), however, the absence of positive peak near 225 nm and the highly negative peak near 210 nm is an indication of random coil which demonstrates that the control peptide lacks triple helical structure.

### Example 4: Peptide Labeling with ^{99m}TcO₄⁻ and Tracer Stability

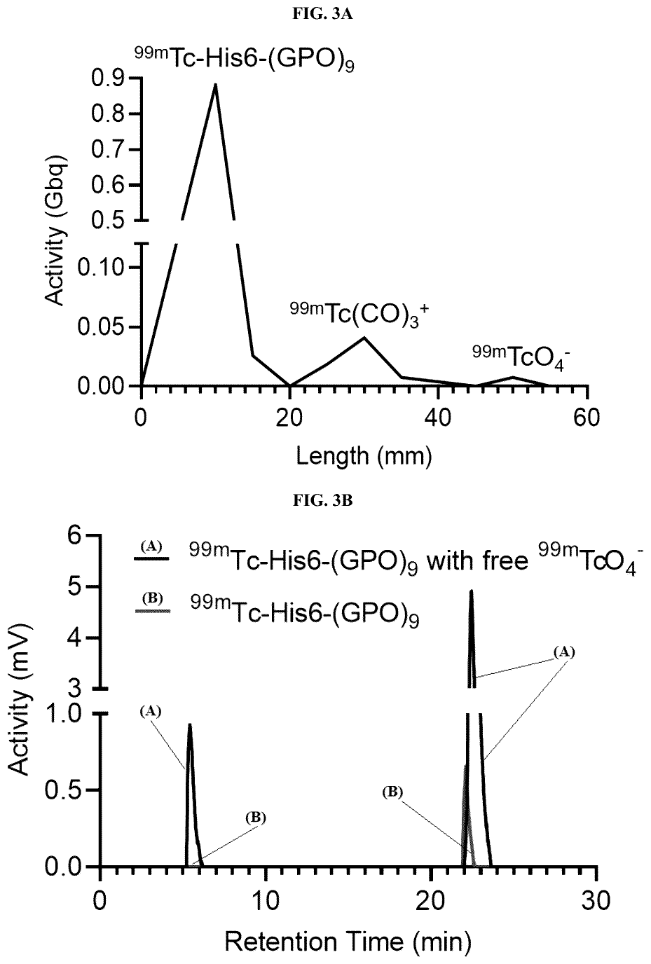

The peptides were labeled with ^{99m}TcO₄⁻ using the technetium tricarbonyl conversion technique known to those skilled in the art, and unbound ^{99m}Tc was separated from the labeled peptide by size-exclusion chromatography. As shown in FIG. 3A, the formation of ^{99m}Tc-bound peptide with a radiolabeling yield of ≥95% was confirmed with radio-HPLC. Free ^{99m}TcO₄⁻ elutes at an earlier time point (˜5 min) compared to ^{99m}Tc-His6-(GPO)₉ (˜22 min). Heating of the tracer to 70° C. for 5 minutes did not affect its stability, and a preliminary in vitro evaluation showed that ^{99m}Tc-His6-(GPO)₉ binds to degraded collagen. The stability of the tracer, ^{99m}Tc-His6-(GPO)₉ in urine was further tested at 2 and 3 hours post injection using radio-HPLC. As shown in FIG. 3B, no free ^{99m}TcO₄⁻ can be detected at 2 and 3 h post injection in urine which is similar to the tracer before injection (0 h) indicating that ^{99m}Tc is bound to the peptide.

### Example 5: Blood Clearance and Biodistribution

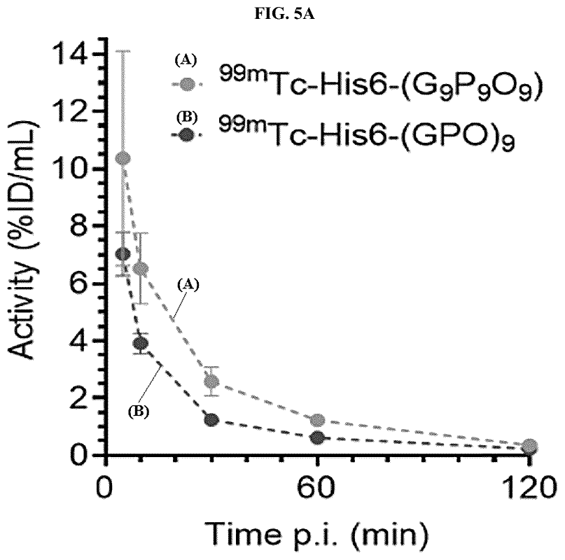

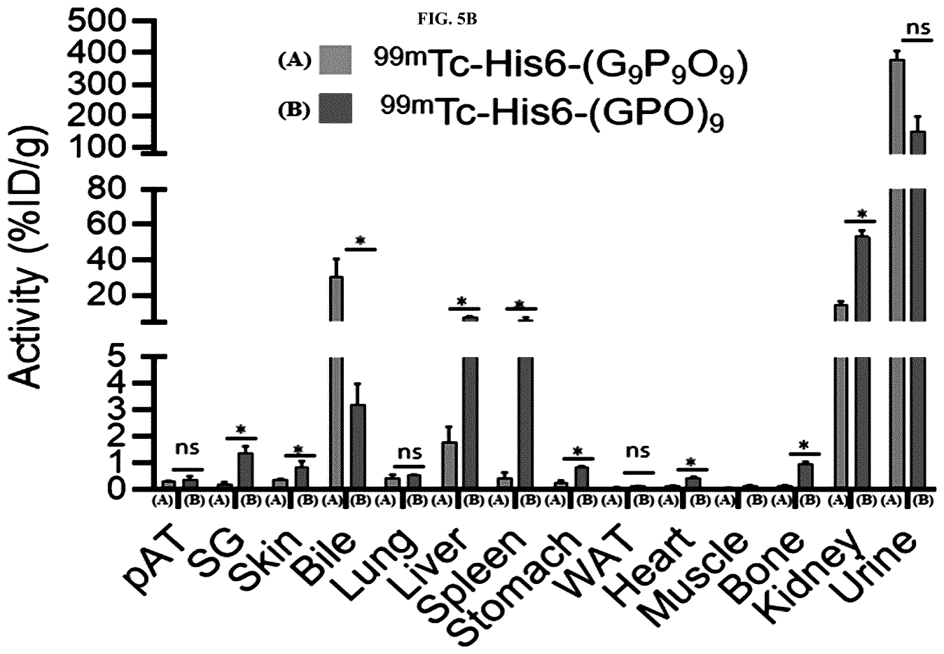

Using gamma counting, tracers' blood levels were measured shown in FIG. 5A at 2 h post injection. Based on the activity corrected by the injection dose, both ^{99m}Tc-His6-(GPO)₉ and control tracer, His6-(G₉P₉O₉) cleared from the blood within 2 hours as no activity was detected at 120 min time point. In addition, biodistribution at 2 hours post injection (p.i.) of ^{99m}Tc-His6-(GPO), and control ^{99m}Tc-His6-(G₉P₉O₉) was assessed by gamma well counting (FIG. 5B, ID: injected dose, pAT: peri-aortic adipose tissue, SG: salivary glands, WAT: white adipose tissue).

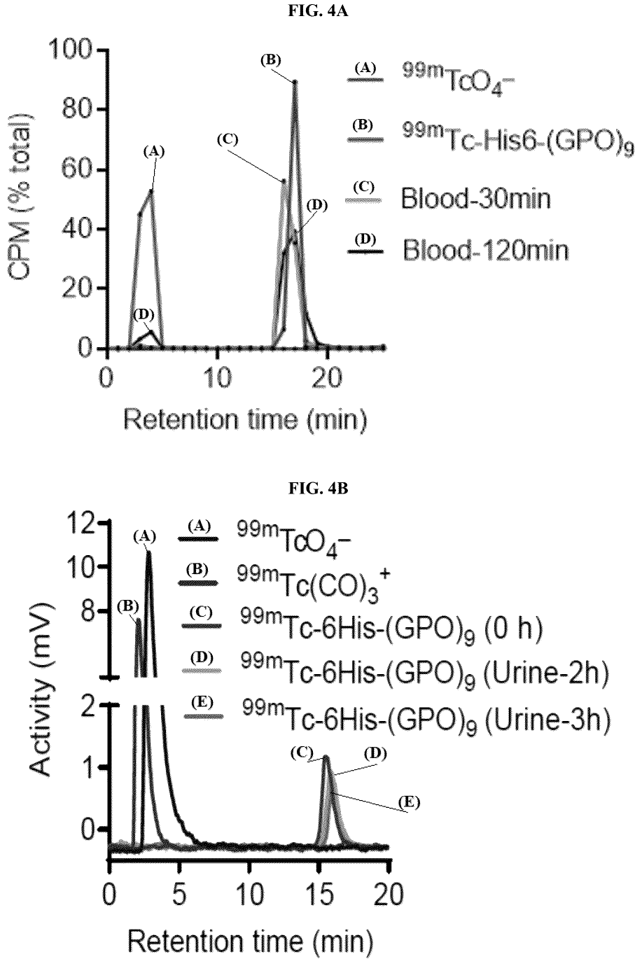

Thus, liver and spleen radioactivity at 2 hours were significantly higher for ^{99m}Tc-His6-(GPO)₉ than the scrambled control tracer (i.e., ^{99m}Tc-His6-(G₉P₉O₉), suggesting that a component of specificity for tracer uptake in these two organs. Further, consistent with reported stability of GPO peptides, 97-99% and 91-94% of ^{99m}Tc-His6-(GPO), remained in the intact form in the mouse blood at 30 minutes and 2 hours post-injection, respectively (FIG. 4A).

### Example 6: Ex Vivo Autoradiography of AAA Using ^{99m}Tc-His6-(GPO)₉

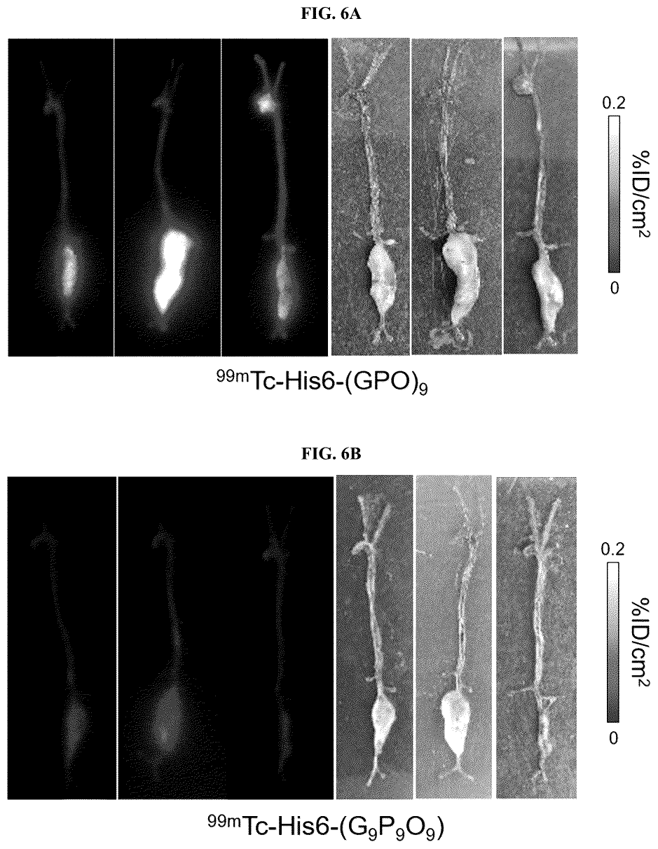

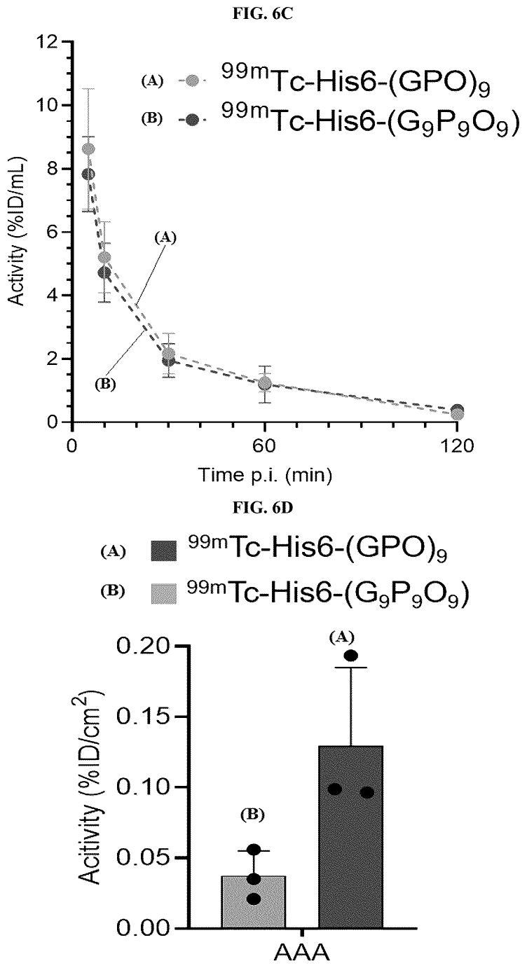

Ex vivo autoradiography was performed at 2 hour post injection of the tracers. As FIGS. 6A-6B and 6D demonstrate, ^{99m}Tc-His6-(GPO), had significantly higher uptake in the aneurysm compared to the control tracer (Left). Of note, ^{99m}Tc-His6-(GPO)₉ and ^{99m}Tc-His6-(G₉P₉O₉) showed comparable, fast blood clearances for up to 2 hr post injection (FIG. 6C).

### Example 7: SPECT/CT Imaging of AAA Using ^{99m}Tc-His6-(GPO)₉

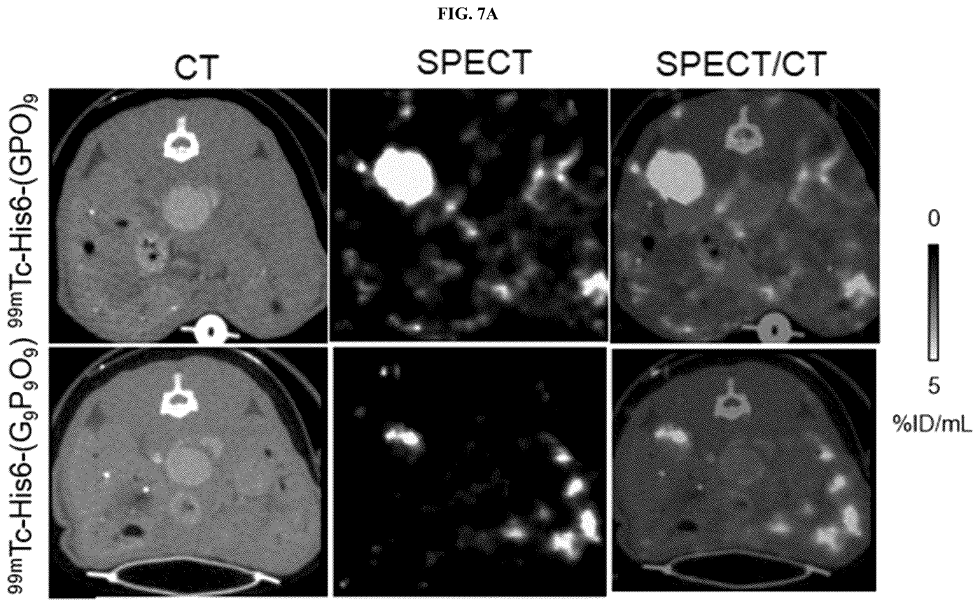

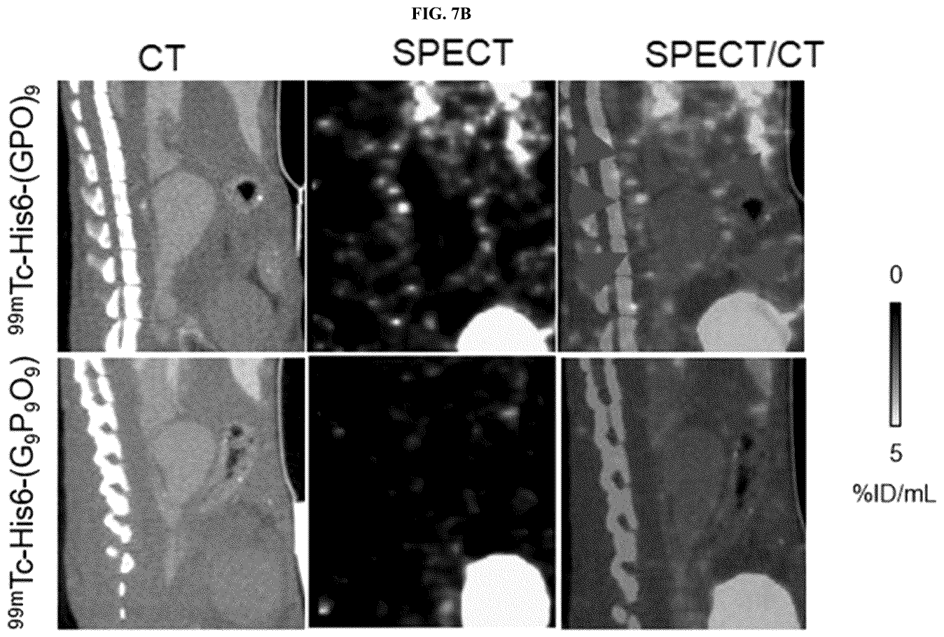

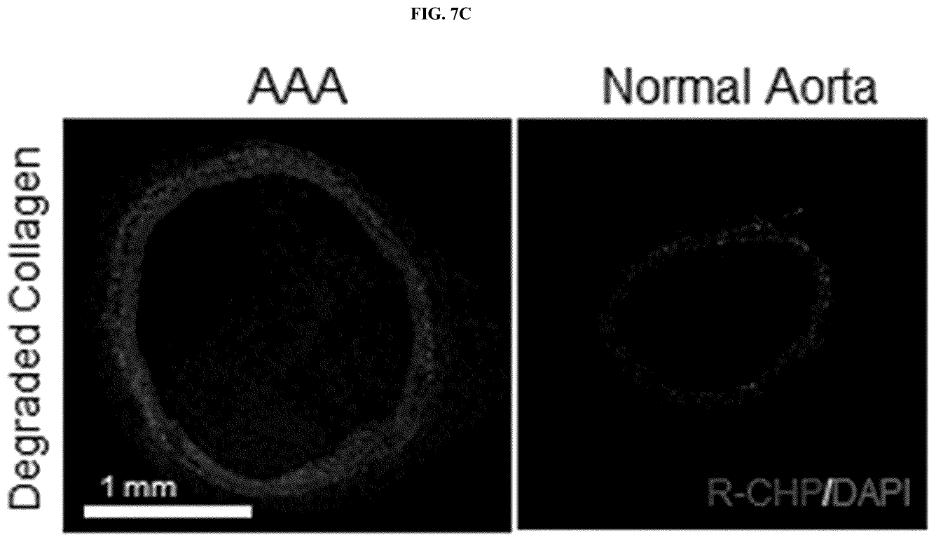

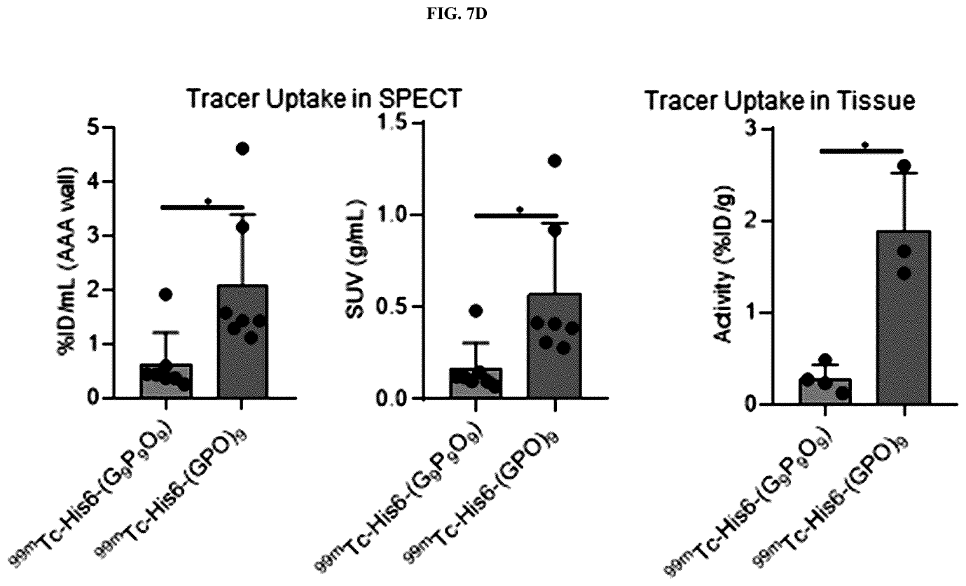

FIGS. 7A-7B shows in vivo SPECT/CT imaging of AAA induced by BAPN/Elastase at 30 days post-surgery. As shown by axial and sagittal images as well as quantification of activity (FIG. 7D), ^{99m}Tc-His6-(GPO), shows higher uptake (red arrow) in the AAA wall compared to the same animal that was injected with ^{99m}Tc-His6-(G₉P₉O₉). Furthermore, the presence of single stranded collagen was confirmed by R-CHP staining (FIG. 7C).

### Example 8: Novel Radiotracer for Imaging of Pulmonary Fibrosis

Pulmonary fibrosis is the characteristic feature in interstitial lung disease (ILD), and affects a growing number of subjects in the US. A subset of patients with interstitial lung disease are affected by idiopathic pulmonary fibrosis, a lethal disease with no cure, but for which new therapies are emerging. Currently, it is not clear how these drugs can be best utilized, and which patients might befit from them. Chronic hypersensitivity pneumonitis and drug (e.g., bleomycin, amiodarone) toxicity are amongst other causes of ILD, and may improve after stopping the causal agent.

The diagnosis of pulmonary fibrosis is based on symptoms (e.g., dyspnea and cough), spirometry, chest radiography and high-resolution computed tomography (CT). In some cases, especially in patients with atypical imaging results, lung biopsy may be necessary to establish the diagnosis. However, this invasive procedure can be associated with major complications, especially in high risk patients, including those requiring supplemental oxygen. CT [and similar structural imaging technologies such as gadolinium-enhanced magnetic resonance imaging (MRI)] provide a snapshot of the lung structure at a given point, without providing any information on disease activity, which is arguably the target of therapeutic interventions to prevent progression and promote regression of fibrosis. In addition, there is some lag before the effect of therapeutic interventions is reflected on the lung structure, and hence becomes detectable by CT or MRI.

Accordingly, novel tools are needed to characterize fibrosis, detect matrix turnover, select the patients for emerging therapies, track the effect of therapeutic interventions to guide the timing of initiation and discontinuation, and improve prognostication.

It is assumed that the process of fibrogenesis in the lungs is triggered by tissue injury. This can lead to dysregulated collagen synthesis and deposition. The hallmark of collagen structure is triple helix, a right-handed helix of 3 α-chains. α-chains are formed by repetitive Gly-X-Y (where X and Y are frequently proline and hydroxyproline) tri-peptide motifs, which self-assemble to form (pro)collagen fibers.

Collagen is initially synthesized as procollagen, a precursor molecule with C- and N-terminal propeptides flanking the Gly-X-Y motifs. Following translation, the procollagen α-chains are imported into the endoplasmic reticulum, where they assemble into triple helix procollagen. Procollagen undergoes proteolytic cleavage to form collagen following its export in to extracellular space, where it assembles into fibrils and higher-ordered structures.

Accordingly, in the mature collagen fibers, the α-chains are highly organized. Single stranded α-chains are only present during collagen synthesis in the cells, and when collagen is undergoing degradation, e.g., by matrix metalloproteinases and cathepsins, in the extracellular space. It was therefore hypothesized that the self-assembly of α-chains into a triple helix can be exploited to track matrix remodeling in pulmonary fibrosis.

The diagnostic gaps in the tools currently used to characterize fibrosis, such as pulmonary fibrosis, may be addressed by in vivo molecular imaging aimed at detecting the biological processes involved in pulmonary fibrosis. Amongst molecular imaging technologies, which rely on targeted tracers, nuclear imaging techniques, such as single photon emission computed tomography (SPECT) and positron emission tomography (PET), are highly sensitive and can provide quantitative data to track the disease process in vivo. PET and SPECT have their own advantages and disadvantages, but SPECT is cheaper and more widely available with well-established radiochemistry techniques.

Several new tracers have emerged in recent years to evaluate fibrosis. These agents target mature collagen, integrins or fibroblasts. A major shortcoming of these targets is that they do not distinguish between established disease and ongoing matrix remodeling which accompanies active fibrogenesis and resolution of fibrosis.

To address this limitation and as a novel approach to imaging pulmonary fibrosis to individualize therapy, new imaging agents were developed, as described herein, to target collagen turnover by taking advantage of collagen self-assembly. It was hypothesized that by targeting collagen turnover, early stages of matrix remodeling in pulmonary fibrosis could be detected. Thus, in one aspect, the present disclosure relates to the development of such a tracer, and validation thereof, in a murine model of pulmonary fibrosis, relying on multi-modality imaging to track fibrosis and its resolution in vivo.

### Example 9: Molecular Imaging of Collagen Turnover in Cardiomyopathy

Heart failure is a major cause of morbidity and mortality worldwide. Cardiomyopathy, which is the pathology that underlies most cases of heart failure, is often triggered by myocardial injury. This injury promotes inflammation, fibroblast proliferation and myofibroblast transformation, and deposit of fibrotic tissue within the myocardium.

Cardiac fibrosis may be classified into reactive interstitial fibrosis and replacement fibrosis. Myocardial infarction (MI) leads to predominantly replacement fibrosis (scar formation) as part of the repair process along with interstitial fibrosis remote from the infarct zone, which promotes ventricular stiffness and maladaptive left ventricle (LV) remodeling. A similar fibrotic process, yet predominantly in the form of interstitial fibrosis, prevails in hypertrophic cardiomyopathy and other forms of cardiac hypertrophy. As such, cardiac fibrosis directly contributes to structural and biological changes that ultimately lead to heart failure and its complications.

Accordingly, the presence and extent of fibrotic tissue have diagnostic and prognostic implications in cardiomyopathy. Structural imaging modalities such as magnetic resonance imaging (MRI) and computed tomography (CT), can provide a snapshot of the cardiac structure at a given point. However, they do not provide any information on the fibrotic process, which is the target of therapeutic interventions to prevent the progression and promote the regression of fibrosis. Therefore, novel non-invasive quantitative tools are needed to characterize fibrosis, detect matrix turnover, select the patients for emerging therapies, track the effect of therapeutic interventions, and improve prognostication.

Many existing gaps in the traditional approach to cardiac imaging may be addressed by molecular imaging. Molecular imaging modalities that rely on radiotracers, i.e., single photon emission computed tomography (SPECT) and positron emission tomography (PET), are highly sensitive and can provide quantitative data to track the disease process in vivo. In recent years, several new tracers have been introduced to detect fibrosis. These agents target mature collagen, integrins or fibroblasts. A major shortcoming of these agents is that they do not distinguish between established disease and ongoing matrix remodeling which accompanies active fibrogenesis and resolution of fibrosis.

Cardiac fibrosis consists mainly of collagen types I and III. During ventricular remodeling, the highly organized mature collagen fibers are degraded by proteases such as matrix metalloproteinases (MMPs) into single stranded α-chains that are not normally present in the extracellular space. Without wishing to be limited by any theory, it was hypothesized that single stranded collagen imaging can track the development and regression of fibrosis in ventricular remodeling. To address this hypothesis, and as a novel approach to imaging cardiac fibrosis, novel radiotracers were developed, as described herein, to target collagen turnover by taking advantage of collagen triple helix self-assembly. This novel class of peptide-based radiotracers is designed with a modular structure for molecular imaging of single stranded collagen, and include a prototype tracer, ^{99m}Tc-His6-(GPO)₉, as described herein, has yielded promising results.

A major limitation of the current probes used to image fibrosis is that they cannot differentiate intact (mature) collagen (e.g., in a scar) from active collagen remodeling. Furthermore, none of the techniques used to detect fibrosis can distinguish between established disease and ongoing matrix remodeling which accompanies active fibrogenesis and resolution of fibrosis, i.e., collagen remodeling (turnover). The loss of myocardial collagen scaffold, which involves the degradation of mature collagen into a SS form, is an early process within the infarct zone. Subsequently, the SS form appears during the fibrotic process, which is associated with collagen turnover, within the infarct (and potentially remote) zone.

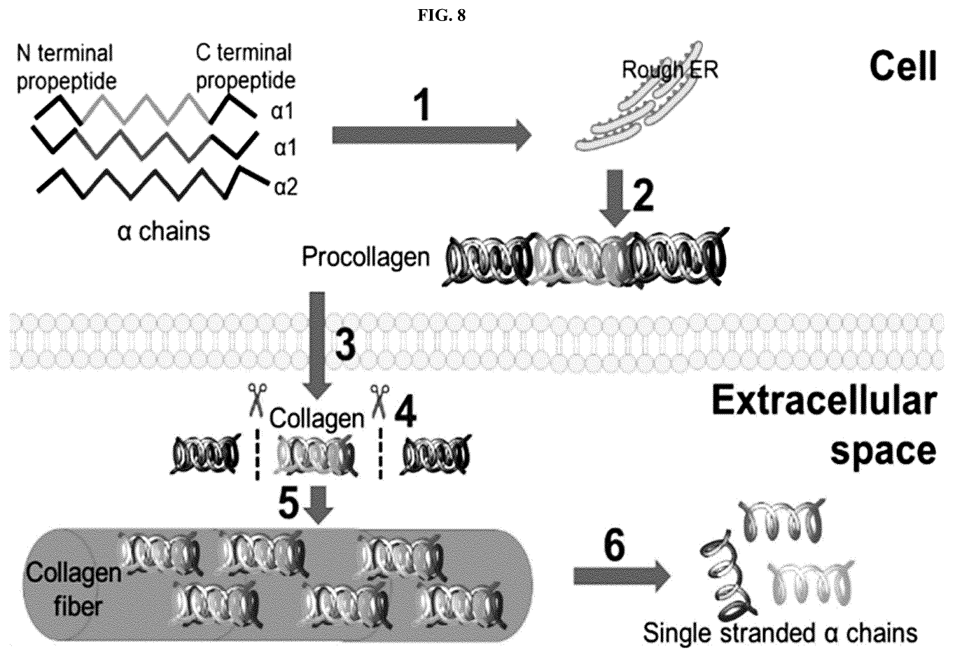

To address this unmet need and as a novel approach to imaging collagen turnover in cardiac fibrosis, this project is focused on novel SPECT radiotracers that target extracellular single-stranded collagen by taking advantage of collagen's triple helix assembly (FIG. 8). This novel class of peptide-based radiotracers is designed with a modular structure, and include a prototype tracer, ^{99m}Tc-His6-(GPO)₉, which has yielded promising results in preliminary studies. Nuclear techniques, PET and SPECT, are highly sensitive and can provide quantitative data to track the disease process in vivo. The focus on SPECT is motivated by its high sensitivity, higher spatial resolution (0.25 mm) for small animal studies, established radiochemistry, ease of use, and a large body of evidence and infrastructure for cardiac imaging which would facilitate clinical translation. While still not as good as clinical PET imaging, the spatial resolution of clinical SPECT scanners is improving with the introduction of Cadmium Zinc Telluride (CZT) detectors and cardiac-dedicated SPECT/CT systems exhibit higher sensitivity compared to whole-body scanners. Nonetheless, the modular design may be adapted to generate copper-64 or F-18-labeled homolog tracers for PET imaging to take advantage of its superior sensitivity and quantitative nature. While the focus here is on cardiac fibrosis and remodeling, the implications of these findings extends to other diseases with fibrosis, such as pulmonary fibrosis, cirrhosis and cancer, where better tools for diagnosis, prognostication and drug development are needed.

### Example 10: Imaging in a Predominantly Replacement Fibrosis Model

To generate a model of predominantly replacement fibrosis, C57BL/6 mice underwent surgical ligation of the left anterior descending (LAD) coronary artery with or without reperfusion to generate different extents of tissue injury. The post-operative survival rate was lower following reperfusion. Therefore, the permanent ligation model (with 89% post-operative survival) was selected for the following studies, with the ischemia-reperfusion model serving as alterative, if needed.

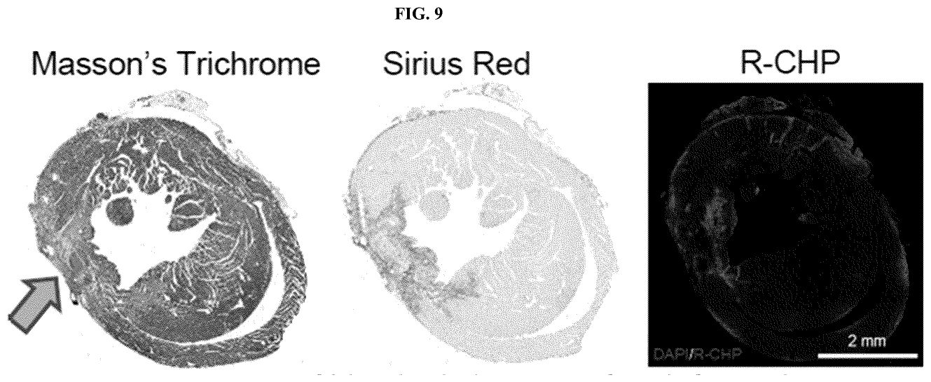

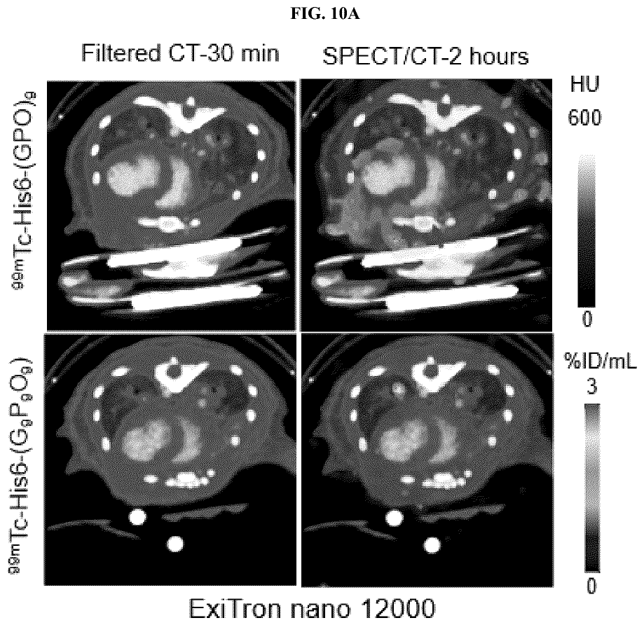

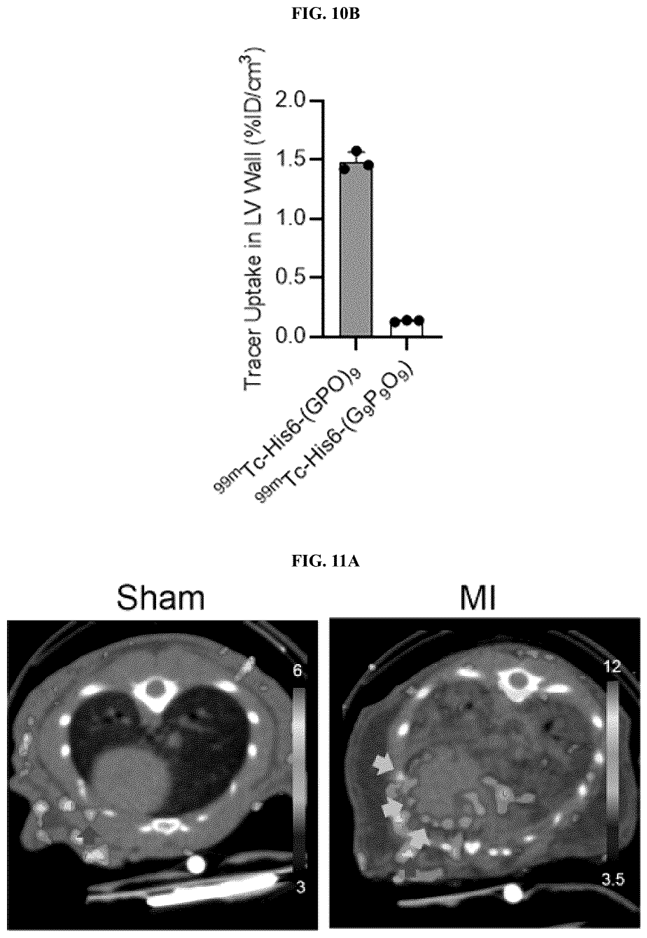

As expected, coronary ligation led to scar formation and considerable LV remodeling over a 4-week period. Importantly, sulfo-Cyanine3 conjugated collagen hybridizing peptide (R-CHP, 3Helix) staining showed the presence of single stranded collagen within the scar, and to a smaller extent, in the remote myocardium (FIG. 9). A preliminary in vivo microSPECT/CT imaging study evaluated the uptake of ^{99m}Tc-His6-(GPO)₉ at 10 days post-MI. In vivo microSPECT/CT imaging study evaluated the uptake of ^{99m}Tc-His6-(GPO), in a murine model of MI induced by left anterior descending coronary artery (LAD)-ligation post days. At 2 hr p.i., the ^{99m}Tc-His6-(GPO), signal was readily detectable in the antero-apical wall of the infarcted heart (FIG. 10A). A scar tissue along with SS collagen (detected using sulfo-Cyanine3 conjugated collagen hybridizing peptide, R-CHP, 3Helix) was present in the anterior wall on tissue sections (FIG. 9). No focal uptake was detectable in a normal heart (FIG. 10A-10B). In a similar study, the ^{99m}Tc-His6-(GPO), signal was readily detectable in the anterior and lateral walls of the infarcted (orange arrows), but not sham-operated, hearts (FIGS. 11A-11B).

### Example 11: Imaging in a Predominantly Interstitial Fibrosis Model

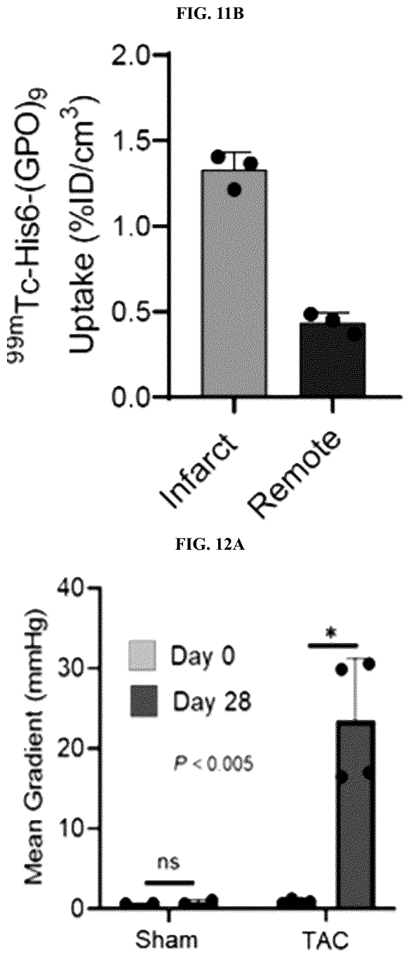

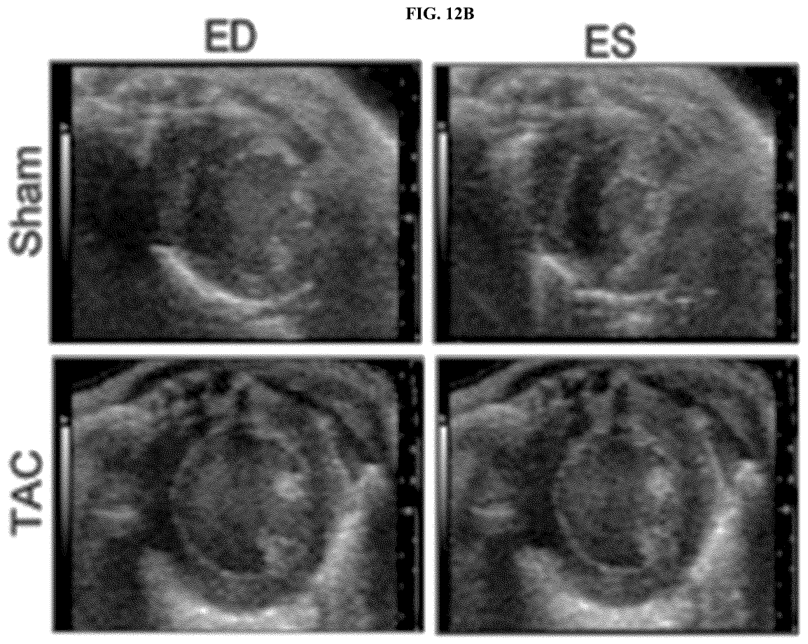

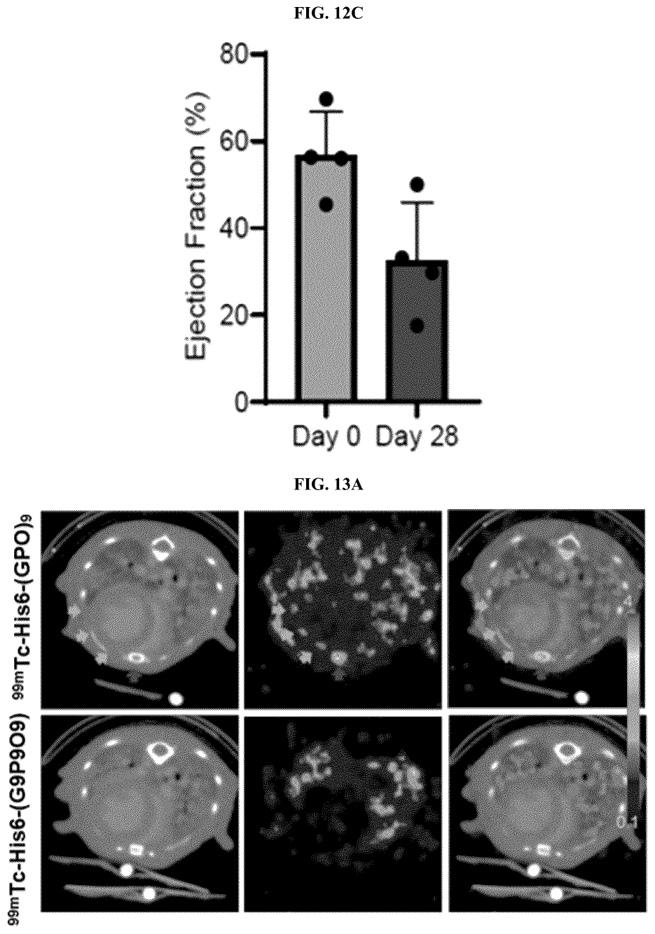

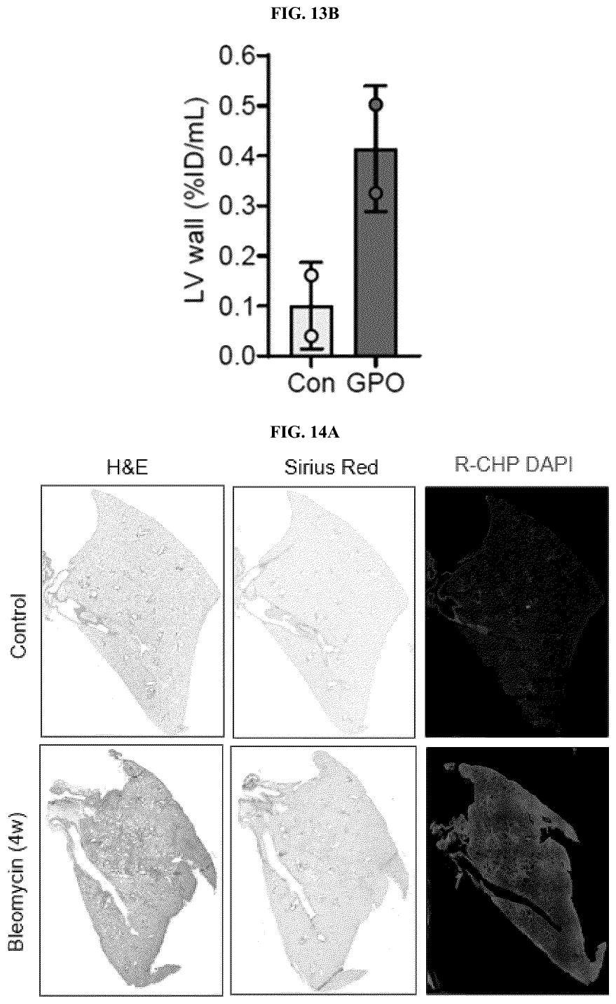

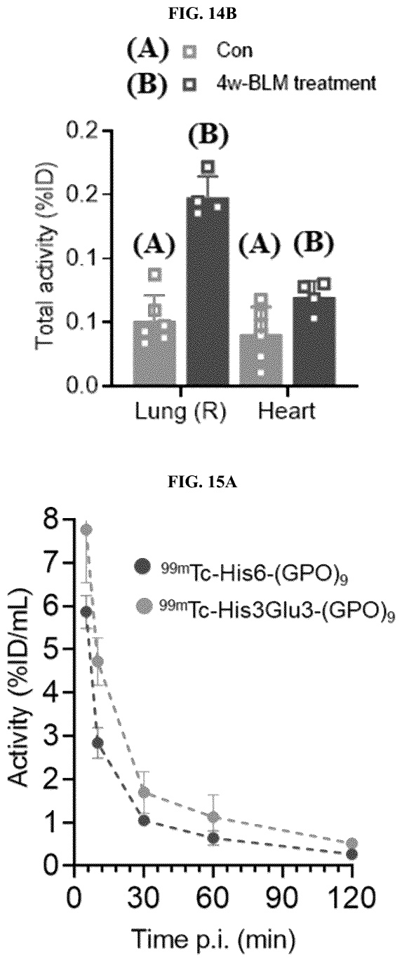

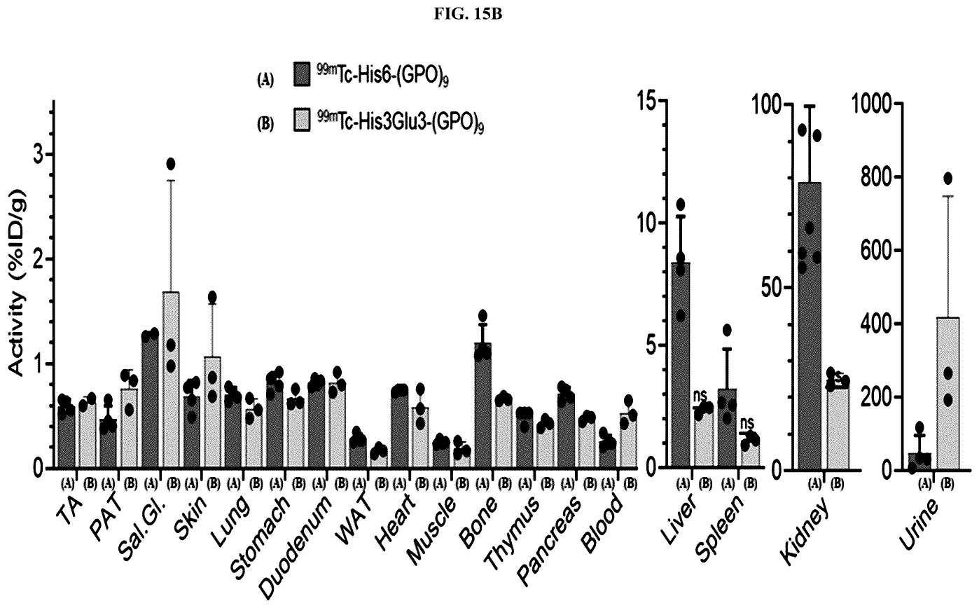

To generate a model of predominantly interstitial fibrosis, C57BL/6 mice underwent transverse aortic constriction (TAC). As expected, this led to considerable LV hypertrophy and dilation, and a reduction in LV ejection fraction by 4 weeks after surgery (FIGS. 12A-12C). Examples of end diastole (ED) and end systole (ES) 2D echo images at 4 weeks after surgery in TAC and sham-operated animals can be seen in FIG. 12B. Preliminary ^{99m}Tc-His6-(GPO)₉ and ^{99m}Tc-His6-(G₉P₉O₉) SPECT/CT studies showed specific uptake of ^{99m}Tc-His6-(GPO)₉ in the remodeled myocardium at 4 weeks after TAC (FIGS. 13A-13B).

| | Sequence Listing |
|---|---|
| SEQ ID NO: 1 | |
| | GGSGG |
| SEQ ID NO: 2 | |
| | GSGS |
| | ((GPO)₉) |
| SEQ ID NO: 3 | |
| | GPOGPOGPOGPOGPOGPOGPOGPOGPO |
| | (G₉P₉O₉) |
| SEQ ID NO: 4 | |
| | GGGGGGGGGPPPPPPPPPOOOOOOOOO |
| | (His6) |
| SEQ ID NO: 5 | |
| | HHHHHH |
| | ((HisGlu)₃) |
| SEQ ID NO: 6 | |
| | HQHQHQ |

### ENUMERATED EMBODIMENTS

The following exemplary embodiments are provided, the numbering of which is not to be construed as designating levels of importance:
Embodiment 1 provides a compound of formula (I):

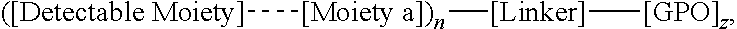

or a salt, solvate, or derivative thereof, wherein
each [Detectable Moiety] is independently a radioisotope or metal selected from the group consisting of ³H, ¹¹C, ¹³C, ¹³N, ¹⁵O, ¹⁸F, ³²P, ³⁵S, ^{99m}Tc, ¹²³I, ⁶⁴Cu, ⁶⁷Ga, ⁶⁸Ga, ¹¹¹In, Gd³⁺, Fe³⁺, Mn²⁺, and Mn³⁺, wherein [Detectable Moiety] is optionally coordinated to one or more ancillary ligands to satisfy its valency;
each [Moiety A] is independently selected from the group consisting of:
   a peptide comprising between 1 and 20 natural or unnatural amino acid residues,
   a polyaminopolycarboxylato-based chelator, and
   an organic compound comprising a leaving atom or a leaving group that can be substituted with a radioisotope:
[Linker] is selected from the group consisting of: a peptide comprising between 1 and 20 natural or unnatural amino acid residues, a small cyclic organic molecule, a small acyclic organic molecule, polyethylene glycol, polypropylene glycol, and a hydrocarbon chain;
[GPO] is glycine-proline-hydroxyproline, wherein each instance of proline is independently optionally (2S,4S)-4-fluoroproline;
n is an integer between 1 and 10;
z is an integer between 2 and 20; and
wherein [symbol] represents one or more coordinate covalent bonds, one or more covalent bonds, or a combination thereof;
and [symbol] represents one or more covalent bonds.
Embodiment 2 provides the compound of claim 1, wherein the one or more ancillary ligands comprise at least one of F⁻, Cl⁻, Br⁻, I⁻, CN⁻, HO—, CO, NO₂⁻, and H₂O.
Embodiment 3 provides the compound of any one of claim 1 or 2, wherein n is 1, [Detectable Moiety] is ^{99m}Tc coordinated to 3 ancillary CO ligands, and [symbol] represents 3 coordinate covalent bonds between [Detectable Moiety] and [Moiety A].
Embodiment 4 provides the compound of any one of claims 1-3, wherein each [Moiety A] is independently selected from the group consisting of:
(i) between 2 and 20 repeating histidine residues,
(ii) (HisGlu)₃.
(iii) (Gly)_{w}Cys, wherein w is an integer from 0 to 20,
(iv) a polyaminopolycarboxylato-based chelator selected from the group consisting of diethylenetriaminepentaacetic acid (DTPA) 1,4,7,10-tetraazacyclododecane-1,4,7,10-tetraacetic acid (DOTA), 1,4,7,10-tetraazacyclododececane, 1-(glutaric acid)-4,7,10-triacetic acid (DOTAGA), ethylenediaminetetraacetic acid (EDTA), 1,4,7-triazacyclononane-triacetic acid (NOTA), 1,4,7-triazacyclononane, 1-glutaric acid-4,7-acetic acid (NODAGA), and 1,4,8,11-tetraazacyclotetradecane-1,4,8,11-tetraacetic acid (TETA), and
(v) an organic compound comprising a leaving atom or a leaving group selected from the group consisting of an aryltrifluoromethane sulfonate and an alkyltrifluoromethane sulfonate.
Embodiment 5 provides the compound of any one of claims 1-4, wherein [Linker] is selected from the group consisting of:
(i) between 1 and 20 repeating glycine residues,
(ii) between 1 and 20 repeating serine residues,
(iii) [G]ₓ[S]_{y}, wherein x and y are each independently an integer selected from 1 to 20,
(iv) GGG, GGSGG, or GSGS
(v) polyethylene glycol or polypropylene glycol, and
(vi) a C₁-C₂₀ hydrocarbon chain.
Embodiment 6 provides the compound of any one of claims 1-5, wherein [Moiety A] is between 2 and 10 histidine residues.
Embodiment 7 provides the compound of any one of claims 1-6, wherein [Moiety A] is six histidine residues.
Embodiment 8 provides the compound of any one of claim 6 or 7, wherein n is 1 and [symbol] represents 3 coordinate covalent bonds between [Detectable Moiety] and the histidine nitrogen atoms of [Moiety A].
Embodiment 9 provides the compound of any one of claims 1-8, wherein [Linker] is GGG.
Embodiment 10 provides the compound of any one of claims 1-9, wherein z is 9.
Embodiment 11 provides the compound of any one of claims 1-10, wherein the compound of formula (I) is ^{99m}Tc-His6-(GPO)₉, wherein ^{99m}Tc is coordinated to 3 CO ligands.
Embodiment 12 provides a method of imaging collagen turnover in a subject, the method comprising at least one of the following steps:
(a) administering to the subject a compound of formula (I):

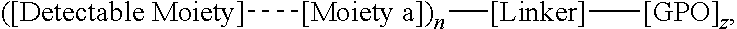

or a salt, solvate, or derivative thereof, wherein
each [Detectable Moiety] is independently a radioisotope or metal selected from the group consisting of ³H, ¹¹C, ¹³C, ¹³N, ¹⁵O, ¹⁸F, ³²P, ³⁵S, ^{99m}Tc, ¹²³I, ⁶⁴Cu, ⁶⁷Ga, ⁶⁸Ga, ¹¹¹In, Gd³⁺, Fe³⁺, Mn²⁺, and Mn³⁺, wherein [Detectable Moiety] is optionally coordinated to one or more ancillary ligands to satisfy its valency;
each [Moiety A] is independently selected from the group consisting of:
   a peptide comprising between 1 and 20 natural or unnatural amino acid residues,
   a polyaminopolycarboxylato-based chelator, and
   an organic compound comprising a leaving atom or a leaving group that can be substituted with a radioisotope:
[Linker] is selected from the group consisting of: a peptide comprising between 1 and 20 natural or unnatural amino acid residues, a small cyclic organic molecule, a small acyclic organic molecule, polyethylene glycol, polypropylene glycol, and a hydrocarbon chain;
[GPO] is glycine-proline-hydroxyproline, wherein each instance of proline is independently optionally (2S,4S)-4-fluoroproline;
n is an integer between 1 and 10;
z is an integer between 2 and 20; and
wherein [symbol] represents one or more coordinate covalent bonds, one or more covalent bonds, or a combination thereof;
and [symbol] represents one or more covalent bonds; and
(b) detecting a signal from the compound within the subject.
Embodiment 13 provides the method of claim 12, wherein the one or more ancillary ligands comprise at least one of F⁻, C⁻, Br⁻, I⁻, CN⁻, HO—, CO, NO₂⁻, and H₂O.
Embodiment 15 provides the method of any one of claim 12 or 13, wherein n is 1, [Detectable Moiety] is ^{99m}Tc coordinated to 3 ancillary CO ligands, and [symbol] represents 3 coordinate covalent bonds between [Detectable Moiety] and [Moiety A].
Embodiment 16 provides the method of any one of claims 12-14, wherein each [Moiety A] is independently selected from the group consisting of:
(i) between 2 and 20 repeating histidine residues,
(ii) (HisGlu)₃.
(iii) (Gly)_{w}Cys, wherein w is an integer from 0 to 20,
(iv) a polyaminopolycarboxylato-based chelator selected from the group consisting of diethylenetriaminepentaacetic acid (DTPA) 1,4,7,10-tetraazacyclododecane-1,4,7,10-tetraacetic acid (DOTA), 1,4,7,10-tetraazacyclododececane, 1-(glutaric acid)-4,7,10-triacetic acid (DOTAGA), ethylenediaminetetraacetic acid (EDTA), 1,4,7-triazacyclononane-triacetic acid (NOTA), 1,4,7-triazacyclononane, 1-glutaric acid-4,7-acetic acid (NODAGA), and 1,4,8,11-tetraazacyclotetradecane-1,4,8,11-tetraacetic acid (TETA), and
(v) an organic compound comprising a leaving atom or a leaving group selected from the group consisting of an aryltrifluoromethane sulfonate and an alkyltrifluoromethane sulfonate.
Embodiment 16 provides the method of any one of claims 12-15, wherein [Linker] is selected from the group consisting of:
(i) between 1 and 20 repeating glycine residues,
(ii) between 1 and 20 repeating serine residues,
(iii) [G]ₓ[S]_{y}, wherein x and y are each independently an integer selected from 1 to 20,
(iv) GGG, GGSGG, or GSGS, and
(v) polyethylene glycol or polypropylene glycol, and
(vi) a C₁-C₂₀ hydrocarbon chain.
Embodiment 17 provides the method of any one of claims 12-16 wherein [Moiety A] is between 2 and 10 histidine residues.
Embodiment 18 provides the method of any one of claims 12-17, wherein [Moiety A] is six histidine residues.
Embodiment 19 provides the method of any one of claim 17 or 18, wherein n is 1 and [symbol] represents 3 coordinate covalent bonds between [Detectable Moiety] and the histidine nitrogen atoms of [Moiety A].
Embodiment 20 provides the method of any one of claims 12-19, wherein [Linker] is GGG.
Embodiment 21 provides the method of any one of claims 12-20, wherein z is 9.
Embodiment 22 provides the method of any one of claims 12-21, wherein the compound of formula (I) is ^{99m}Tc-His6-(GPO)₉, wherein ^{99m}Tc is coordinated to 3 CO ligands.
Embodiment 23 provides the method of any one of claims 12-22, wherein the signal is detected using single photon emission computed tomography (SPECT) imaging, positron emission tomography (PET), magnetic resonance imaging (MRI), magnetic resonance spectroscopic imaging (MRSI), fluorescence imaging, or a combination thereof.
Embodiment 24 provides the method of any one of claims 12-23, wherein the subject has fibrosis or is suspected of having fibrosis associated with a fibrotic disease or disorder.
Embodiment 25 provides the method of claim 24, wherein the fibrotic disease or disorder is pulmonary fibrosis, liver fibrosis, kidney fibrosis, aortic aneurysms, myocardial infarction, cardiomyopathy, scleroderma, heart failure, or a combination thereof.
Embodiment 26 provides the method of any one of claim 24 or 25, further comprising:

administering a treatment for the fibrotic disease or disorder to the subject before step (a); and/ond/or administering a treatment for the fibrotic disease or disorder to the subject after step (b).
Embodiment 27 provides the method of claim 26, wherein the treatment is administered to the subject after step (b) and wherein the method further comprises the steps of:
(c) administering to the subject a compound of formula (I); and
(d) detecting a signal from the compound of formula (I).
Embodiment 28 provides the method of claim 27, wherein a change in the signal from the compound of formula (I) in step (d) compared to step (b) is used to evaluate the effectiveness of the treatment.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the disclosure. The appended claims are intended to be construed to include all such embodiments and equivalent variations.

1. A compound of formula (I): 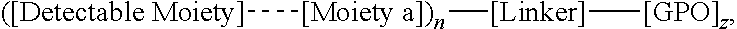
or a salt, solvate, or derivative thereof, wherein;
each [Detectable Moiety] is independently a radioisotope or metal selected from the group consisting of ³H, ¹¹C, ¹³C, ¹³N, ¹⁵O, ¹⁸F, ³²P, ³⁵S, ^{99m}Tc, ¹²³I, ⁶⁴Cu, ⁶⁷Ga, ⁶⁸Ga, ¹¹¹In, Gd³⁺, Fe³⁺, Mn²⁺, and Mn³⁺,
wherein each [Detectable Moiety] is independently optionally coordinated to one or more ancillary ligands to satisfy its valency;
each [Moiety A] is independently selected from the group consisting of:
a peptide comprising 1, 2, 3, 4, 5, 6, 7, 8, 9, 10, 11, 12, 13, 14, 15, 16, 17, 18, 19 or 20 independently selected natural or unnatural amino acid residues,
a polyaminopolycarboxylato-based chelator, and
an organic compound comprising a leaving atom or a leaving group that can be substituted with a radioisotope;
[Linker] is selected from the group consisting of: a peptide comprising 1, 2, 3, 4, 5, 6, 7, 8, 9, 10, 11, 12, 13, 14, 15, 16, 17, 18, 19, or 20 independently selected natural or unnatural amino acid residues, a small cyclic organic molecule, a small acyclic organic molecule, polyethylene glycol, polypropylene glycol, and a hydrocarbon chain;
[GPO] is glycine-proline-hydroxyproline, wherein each instance of proline is independently optionally (2S,4S)-4-fluoroproline;
n is 1, 2, 3, 4, 5, 6, 7, 8, 9, or 10;
z is 2, 3, 4, 5, 6, 7, 8, 9, 10, 11, 12, 13, 14, 15, 16, 17, 18, 19, or 20,
optionally wherein z is 9; and
wherein each [symbol] independently represents one or more coordinate bonds, one or more covalent bonds, or a combination thereof;
and each [symbol] independently represents one or more covalent bonds.

2. The compound of claim 1, wherein the one or more ancillary ligands comprise at least one of F⁻, C⁻, Br⁻, I⁻, CN⁻, HO—, CO, NO₂⁻, and H₂O.

3. The compound of claim 1, wherein at least one of the following applies:
(a) n is 1, [Detectable Moiety] is ^{99m}Tc coordinated to 3 ancillary CO ligands, and [symbol] represents 3 coordinate bonds between [Detectable Moiety] and [Moiety A];
(b) n is 1 and [symbol] represents 3 coordinate covalent bonds between [Detectable Moiety] and the histidine nitrogen atoms of [Moiety A].

4. The compound of claim 1, wherein each [Moiety A] is independently selected from the group consisting of:
(i) 2, 3, 4, 5, 6, 7, 8, 9, 10, 11, 12, 13, 14, 15, 16, 17, 18, 19, or 20 repeating histidine residues,
optionally wherein each [Moiety A] is independently 2, 3, 4, 5, 6, 7, 8, 9, or 10 histidine residues,
(ii) (HisGlu)₃,
(iii) (Gly)_{w}Cys, wherein w is 0, 1, 2, 3, 4, 5, 6, 7, 8, 9, 10, 11, 12, 13, 14, 15, 16, 17, 18, 19, or 20,
(iv) a polyaminopolycarboxylato-based chelator selected from the group consisting of diethylenetriaminepentaacetic acid (DTPA) 1,4,7,10-tetraazacyclododecane-1,4,7,10-tetraacetic acid (DOTA), 1,4,7,10-tetraazacyclododececane, 1-(glutaric acid)-4,7,10-triacetic acid (DOTAGA), ethylenediaminetetraacetic acid (EDTA), 1,4,7-triazacyclononane-triacetic acid (NOTA), 1,4,7-triazacyclononane, 1-glutaric acid-4,7-acetic acid (NODAGA), and 1,4,8,11-tetraazacyclotetradecane-1,4,8,11-tetraacetic acid (TETA), and
(v) an organic compound comprising a leaving atom or a leaving group selected from the group consisting of an aryltrifluoromethane sulfonate and an alkyltrifluoromethane sulfonate.

5. The compound of claim 1, wherein [Linker] is selected from the group consisting of:
(i) 1, 2, 3, 4, 5, 6, 7, 8, 9, 10, 11, 12, 13, 14, 15, 16, 17, 18, 19, or 20 repeating glycine residues,
(ii) 1, 2, 3, 4, 5, 6, 7, 8, 9, 10, 11, 12, 13, 14, 15, 16, 17, 18, 19, or 20 repeating serine residues,
(iii) [G]ₓ[S]_{y}, wherein x and y are each independently 1, 2, 3, 4, 5, 6, 7, 8, 9, 10, 11, 12, 13, 14, 15, 16, 17, 18, 19, or 20,
(iv) GGG, GGSGG, or GSGS, optionally wherein the [Linker] is GGG,
(v) polyethylene glycol or polypropylene glycol, and
(vi) a C₁-C₂₀ hydrocarbon chain.

6. The compound of claim 1, wherein the Linker is a peptide which N-terminus is covalently bound to the C-terminus of the [GPO]_{z}.

7. The compound of claim 1, wherein the Linker is a peptide which C-terminus is covalently bound to the N-terminus of the [GPO]_{z}.

8-10. (canceled)

11. The compound of claim 1, wherein the compound of formula (I) is ^{99m}Tc-His6-(GPO)₉, wherein ^{99m}Tc is coordinated to 3 CO ligands.

12. A method of imaging collagen turnover in a subject, the method comprising:
(a) administering to the subject a compound of formula (I): 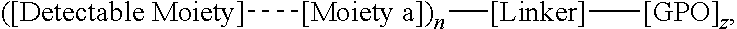
or a salt, solvate, or derivative thereof, wherein
each [Detectable Moiety] is independently a radioisotope or metal selected from the group consisting of ³H, ¹¹C, ¹³C, ¹³N, ¹⁵O, ¹⁸F, ³²P, ³⁵S, ^{99m}Tc, ¹²³I, ⁶⁴Cu, ⁶⁷Ga, ⁶⁸Ga, ¹¹¹In, Gd³⁺, Fe³⁺, Mn²⁺, and Mn³⁺,
wherein each [Detectable Moiety] is independently optionally coordinated to one or more ancillary ligands to satisfy its valency;
each [Moiety A] is independently selected from the group consisting of:
a peptide comprising 1, 2, 3, 4, 5, 6, 7, 8, 9, 10, 11, 12, 13, 14, 15, 16, 17, 18, 19, or 20 independently selected natural or unnatural amino acid residues,
a polyaminopolycarboxylato-based chelator, and
an organic compound comprising a leaving atom or a leaving group that can be substituted with a radioisotope;
[Linker] is selected from the group consisting of: a peptide comprising 1, 2, 3, 4, 5, 6, 7, 8, 9, 10, 11, 12, 13, 14, 15, 16, 17, 18, 19, or 20 independently selected natural or unnatural amino acid residues, a small cyclic organic molecule, a small acyclic organic molecule, polyethylene glycol, polypropylene glycol, and a hydrocarbon chain;
[GPO] is glycine-proline-hydroxyproline, wherein each instance of proline is independently optionally (2S,4S)-4-fluoroproline;
n is 1, 2, 3, 4, 5, 6, 7, 8, 9, or 10;
z is 2, 3, 4, 5, 6, 7, 8, 9, 10, 11, 12, 13, 14, 15, 16, 17, 18, 19, or 20,
optionally wherein z is 9; and
wherein each [symbol] independently represents one or more coordinate bonds, one or more covalent bonds, or a combination thereof; and
each [symbol] independently represents one or more covalent bonds; and
(b) detecting a signal from the compound within the subject.

13. The method of claim 12, wherein the one or more ancillary ligands comprise at least one of F⁻, C⁻, Br⁻, I⁻, CN⁻, HO—, CO, NO₂⁻, and H₂O.

14. The method of claim 12, wherein at least one of the following applies:
(a) n is 1, [Detectable Moiety] is ^{99m}Tc coordinated to 3 ancillary CO ligands, and [symbol] represents 3 coordinate bonds between [Detectable Moiety] and [Moiety A];
(b) n is 1 and [symbol] represents 3 coordinate covalent bonds between [Detectable Moiety] and the histidine nitrogen atoms of [Moiety A].

15. The method of claim 12, wherein each [Moiety A] is independently selected from the group consisting of:
(i) 2, 3, 4, 5, 6, 7, 8, 9, 10, 11, 12, 13, 14, 15, 16, 17, 18, 19, or 20 repeating histidine residues,
optionally wherein each [Moiety A] is independently 2, 3, 4, 5, 6, 7, 8, 9, or 10 histidine residues,
(ii) (HisGlu)₃,
(iii) (Gly)_{w}Cys, wherein w is 0, 1, 2, 3, 4, 5, 6, 7, 8, 9, 10, 11, 12, 13, 14, 15, 16, 17, 18, 19, or 20,
(iv) a polyaminopolycarboxylato-based chelator selected from the group consisting of diethylenetriaminepentaacetic acid (DTPA) 1,4,7,10-tetraazacyclododecane-1,4,7,10-tetraacetic acid (DOTA), 1,4,7,10-tetraazacyclododececane, 1-(glutaric acid)-4,7,10-triacetic acid (DOTAGA), ethylenediaminetetraacetic acid (EDTA), 1,4,7-triazacyclononane-triacetic acid (NOTA), 1,4,7-triazacyclononane, 1-glutaric acid-4,7-acetic acid (NODAGA), and 1,4,8,11-tetraazacyclotetradecane-1,4,8,11-tetraacetic acid (TETA), and
(v) an organic compound comprising a leaving atom or a leaving group selected from the group consisting of an aryltrifluoromethane sulfonate and an alkyltrifluoromethane sulfonate.

16. The method of claim 12, wherein [Linker] is selected from the group consisting of:
(i) 1, 2, 3, 4, 5, 6, 7, 8, 9, 10, 11, 12, 13, 14, 15, 16, 17, 18, 19, or 20 repeating glycine residues,
(ii) 1, 2, 3, 4, 5, 6, 7, 8, 9, 10, 11, 12, 13, 14, 15, 16, 17, 18, 19, or 20 repeating serine residues,
(iii) [G]ₓ[S]_{y}, wherein x and y are each independently 1, 2, 3, 4, 5, 6, 7, 8, 9, 10, 11, 12, 13, 14, 15, 16, 17, 18, 19, or 20,
(iv) GGG, GGSGG, or GSGS, optionally wherein the [Linker] is GGG,
(v) polyethylene glycol or polypropylene glycol, and
(vi) a C₁-C₂₀ hydrocarbon chain.

17. The method of claim 12, wherein the Linker is a peptide which N-terminus is covalently bound to the C-terminus of the [GPO]_{z}.

18. The method of claim 12, wherein the Linker is a peptide which C-terminus is covalently bound to the N-terminus of the [GPO]_{z}.

19-21. (canceled)

22. The method of claim 12, wherein the compound of formula (I) is ^{99m}Tc-His6-(GPO)₉, wherein ^{99m}Tc is coordinated to 3 CO ligands.

23. The method of claim 12, wherein the signal is detected using single photon emission computed tomography (SPECT) imaging, positron emission tomography (PET), magnetic resonance imaging (MRI), magnetic resonance spectroscopic imaging (MRSI), fluorescence imaging, or a combination thereof.

24. The method of claim 12, wherein the subject has fibrosis or is suspected of having fibrosis associated with a fibrotic disease or disorder.

25. The method of claim 24, wherein the fibrotic disease or disorder is pulmonary fibrosis, liver fibrosis, kidney fibrosis, aortic aneurysms, myocardial infarction, cardiomyopathy, scleroderma, heart failure, or a combination thereof.

26. The method of claim 24 , further comprising:
administering a treatment for the fibrotic disease or disorder to the subject before step (a); or
administering a treatment for the fibrotic disease or disorder to the subject after step (b).

27. The method of claim 26, wherein the treatment is administered to the subject after step (b) and wherein the method further comprises the steps of:
(c) administering to the subject a compound of formula (I), or a salt, solvate, or derivative thereof; and
(d) detecting a signal from the compound of formula (I), or a salt, solvate, or derivative thereof.

28. The method of claim 27, wherein a change in the signal from the compound of formula (I) in step (d) compared to step (b) is used to evaluate the effectiveness of the treatment.


SEQUENCE LISTING (6 sequences)

SEQ ID NO: 1 - protein, 5 aa, Artificial Sequence
  Chemically synthesized
GGSGG

SEQ ID NO: 2 - protein, 4 aa, Artificial Sequence
  Chemically synthesized
GSGS

SEQ ID NO: 3 - protein, 27 aa, Artificial Sequence
  Chemically synthesized
  MOD_RES (3)..(3): 4Hyp
  MOD_RES (6)..(6): 4Hyp
  MOD_RES (9)..(9): 4Hyp
  MOD_RES (12)..(12): 4Hyp
  MOD_RES (15)..(15): 4Hyp
  MOD_RES (18)..(18): 4Hyp
  MOD_RES (21)..(21): 4Hyp
  MOD_RES (24)..(24): 4Hyp
  MOD_RES (27)..(27): 4Hyp
GPPGPPGPPGPPGPPGPPGPPGPPGPP

SEQ ID NO: 4 - protein, 27 aa, Artificial Sequence
  Chemically synthesized
  MOD_RES (19)..(27): 4Hyp
GGGGGGGGGPPPPPPPPPPPPPPPPPP

SEQ ID NO: 5 - protein, 6 aa, Artificial Sequence
  Chemically synthesized
HHHHHH

SEQ ID NO: 6 - protein, 6 aa, Artificial Sequence
  Chemically synthesized
HQHQHQ